(19) United States
(12) Patent Application Publication
(10) Pub. No.: US 2021/0395828 A1
(43) Pub. Date: Dec. 23, 2021

(54) METHYLATION MODIFICATION-BASED TUMOR MARKER STAMP-EP1

(71) Applicant: SHANGHAI EPIPROBE BIOTECHNOLOGY CO., LTD., Shanghai (CN)

(72) Inventor: Zhenyan LI, Shanghai (CN)

(21) Appl. No.: 17/250,457

(22) PCT Filed: Jul. 19, 2019
(86) PCT No.: PCT/CN2019/096793

§ 371 Date: Sep. 13, 2021

(30) Foreign Application Priority Data
Jul. 26, 2018 (CN) 201810830893.0

(51) Int. Cl. C12Q 1/6886; C12Q 1/6806

(52) U.S. Cl. CPC C12Q 1/6886; C12Q 2600/154; C12Q 1/6806

(57) ABSTRACT

A methylated tumor marker STAMP-EP1 and a use thereof, which relate to the field of molecular biology. The present disclosure further relates to a use of the methylated tumor marker STAMP-EP1 in the preparation of tumor diagnostic agents. The tumor marker STAMP-EP1 herein is hypermethylated in all tumor types, is hypomethylated in corresponding normal tissue, and with very high sensitivity and specificity; primers for detecting STAMP-EP1 may be used to prepare a tumor diagnostic kit.

### REFERENCE TO A SEQUENCE LISTING SUBMITTED ELECTRONICALLY VIA EFS-WEB

The content of the electronically submitted sequence listing in ASCII text file (Name: 4790_0010001_Seqlisting_ST25; Size: 16,306 bytes; and Date of Creation: Apr. 22, 2021) is herein incorporated by reference in its entirety.

### FIELD OF DISCLOSURE

The disclosure is in the field of disease diagnostic markers. More specifically, the disclosure relates to a methylation based tumor marker STAMP, Specific Tumor Aligned Methylation of Pan-cancer.

### BACKGROUND OF DISCLOSURE

The occurrence and development of tumor is a complex, multi-level, multi-factor dynamic process, including the complex interaction of external environment, genetic variations and epigenetic changes, etc. Environmental factors include carcinogenic physical, chemical, biological and other factors as well as unhealthy living habits. Genetic variations include gene mutation, copy number variation, chromosome translocation and so on. Epigenetic changes mainly include DNA methylation, histone modification, non-coding RNA and other factors. In the process of tumor occurrence and development, environmental, genetic and epigenetic factors complement each other and act together, leading to a series of inactivations of tumor suppressor genes and activations of proto-oncogenes, thereby causing tumor. The three factors act throughout the development of tumor and interact with each other. For human beings, there are still many challenges for tumor therapy at present. Although new surgical methods, targeted therapy and immunotherapy have made some gratifying progress in recent years, there are still many misconceptions about tumor. The major problems of tumor metastasis, recurrence, heterogeneity and drug resistance need to be solved urgently.

There are many types of tumors in human body, and the occurrences of tumors can be found in most of the tissues. Different types of tumors can be divided into many subtypes. Especially in recent years, the classification of tumors is more detailed due to the progress in tumor molecular biology. For different types, different stages or different molecular subtypes of tumors, the therapeutic regimes are also different.

With the deepening of the understanding in tumor and the progress of science and technology, many new tumor markers have been found and used in clinical diagnosis. Before 1980, tumor markers were mainly hormones, enzymes, proteins and other cell secretions, such as carcinoembryonic antigen (CEA) and alpha fetoprotein (AFP) used as markers of gastric cancer, liver cancer and other tumors, carbohydrate antigen 125 (CA125) used as a marker of cervical cancer, and prostate specific antigen (PSA) used as a marker of prostate cancer. Although these tumor markers are still used in clinic, their sensitivity and accuracy can not meet the clinical needs.

Fluid biopsy is a technique for the diagnosis and prediction of tumors using circulating tumor cells or circulating tumor DNA as detection targets. The technology is still in its infancy, having many shortcomings. First, the sensitivity and specificity are not good enough. The tumor itself is heterogeneous, including a variety of subtypes of cell populations. The proportion of tumor DNA in clinical samples, especially blood samples, is very low. The existing tumor markers are difficult to meet the sensitivity of clinical requirements, and it is easy to cause misdiagnosis. Second, one marker has good effect only for one or a few kinds of tumors. As the DNA in blood are very complex, the existing tumor markers cannot solve the complex problems of tumor source and metastasis. Because of these complexities, it is difficult for many DNA methylation tumor markers to have a unified standard in clinical application, which seriously affects the sensitivity and accuracy of the markers. Human tumors have both characteristics and commonness. A common marker for different tumors is of great significance for tumor screening, diagnosis, treatment and efficacy evaluation.

Therefore, it is urgent to develop new tumor markers with general applicability, high accuracy and allowing easy judgment in tumor diagnosis.

### SUMMARY OF DISCLOSURE

The object of the disclosure is to provide a method for detecting tumor based on abnormal hypermethylation of specific sites in tumor using DNA methylation modification as tumor marker.

The first aspect of the present disclosure provides an isolated polynucleotide, including: (a) a polynucleotide with a nucleotide sequence as shown in SEQ ID NO: 1; (b) a fragment of the polynucleotide of (a), having at least one (such as 2-178, more specifically 3, 5, 10, 15, 20, 25, 30, 50, 60, 80, 100, 120, 150) CpG site with modification; and/or (c) a nucleic acid (such as the polynucleotide with a nucleotide sequence as shown in SEQ ID No: 3) complementary to the polynucleotide or fragment of (a) or (b).

In a preferable embodiment, said modification includes 5-methylation, 5-hydroxymethylation, 5-formylcytosine (5fC) or 5-carboxylcytosine (5-caC).

The second aspect of the disclosure provides an isolated polynucleotide, which is converted from the polynucleotide of the first aspect, and as compared with the sequence of the first aspect, the cytosine C of the CpG site(s) with modification is unchanged, and the unmodified cytosine is converted into T or U.

In a preferable embodiment, it is converted from the polynucleotide corresponding to the first aspect by bisulfite treatment. In another preferable embodiment, the polynucleotide includes: (d) a polynucleotide with a nucleotide sequence as shown in SEQ ID NO: 2 or 4; (e) a fragment of the polynucleotide of (d), having at least one (such as 2-178, more specifically 3, 5, 10, 15, 20, 25, 30, 50, 60, 80, 100, 120, 150) CpG site with modification.

The third aspect of the disclosure provides a use of the polynucleotide described in the first or second aspect in manufacture of a tumor detection agent or kit.

In a preferable embodiment, the tumors include (but are not limited to): hematologic cancers such as leukemia, lymphoma, multiple myeloma; digestive system tumors such as esophageal cancer, gastric cancer, colorectal cancer, liver cancer, pancreatic cancer, bile duct and gallbladder cancer; respiratory system tumors such as lung cancer, pleuroma; nervous system tumors such as glioma, neuroblastoma, meningioma; head and neck tumors such as oral cancer, tongue cancer, laryngeal cancer, nasopharyngeal cancer; gynecological and reproductive system tumors such as breast cancer, ovarian cancer, cervical cancer, vulvar cancer, testicular cancer, prostate cancer, penile cancer; urinary system tumors such as kidney cancer, bladder cancer, skin and other systems tumors such as skin cancer, melanoma, osteosarcoma, liposarcoma, thyroid cancer.

In another preferable embodiment, samples of the tumor include but are not limited to: tissue samples, paraffin embedded samples, blood samples, pleural effusion samples, and alveolar lavage fluid samples, ascites and lavage fluid samples, bile samples, stool samples, urine samples, saliva samples, sputum samples, cerebrospinal fluid samples, cell smear samples, cervical scraping or brushing samples, tissue and cell biopsy samples.

The fourth aspect of the disclosure provides a method of preparing a tumor detection agent, including: providing the polynucleotide described in the first or second aspect, designing a detection agent for specifically detecting the modification on CPG site(s) of a target sequence which is the full length or fragment of the polynucleotide; wherein, the target sequence has at least one (such as 2-178, more specifically, 3, 5, 10, 15, 20, 25, 30, 50, 60, 80, 100, 120, 150) modified CpG site; preferably, the detection agent includes (but is not limited to) primers or probes.

The fifth aspect of the disclosure provides an agent or a combination of agents which specifically detect the modification on CPG site(s) of a target sequence, which is the full length or fragment of any of the polynucleotides described in the first or second aspect and has at least one (such as 2-178, more specifically, 3, 5, 10, 15, 20, 25, 30, 50, 60, 80, 100, 120, 150) modified CpG site.

In a preferable embodiment, the agent or combination of agents is for a gene sequence containing the target sequence (designed based on the gene sequence), and the gene sequence includes gene Panels or gene groups.

In another preferable embodiment, the detection agent comprises: primers or probes.

In another preferable embodiment, the primers are: the primers shown in SEQ ID NO: 3 and 4; the primers shown in SEQ ID NO: 7 and 8; the primers shown in SEQ ID NO: 9 and 10; the primers shown in SEQ ID NO: 11 and 12; or the primers shown in SEQ ID NO: 13 and 14.

In the sixth aspect of the disclosure, a use of the agent or combination of agents described in the fifth aspect of the disclosure in the manufacture of a kit for detecting tumors is provided; preferably, the tumors include (but are not limited to): digestive system tumors such as esophageal cancer, gastric cancer, colorectal cancer, liver cancer, pancreatic cancer, bile duct and gallbladder cancer; respiratory system tumors such as lung cancer, pleuroma; hematologic cancers such as leukemia, lymphoma, multiple myeloma; gynecological and reproductive system tumors such as breast cancer, ovarian cancer, cervical cancer, vulvar cancer, testicular cancer, prostate cancer, penile cancer; nervous system tumors such as glioma, neuroblastoma, meningioma; head and neck tumors such as oral cancer, tongue cancer, laryngeal cancer, nasopharyngeal cancer; urinary system tumors such as kidney cancer, bladder cancer, skin and other systems tumors such as skin cancer, melanoma, osteosarcoma, liposarcoma, thyroid cancer.

The seventh aspect of the present disclosure provides a detection kit, comprising container(s) and the agent or combination of agents described above in the container(s); preferably, each agent is placed in an independent container.

In a preferable embodiment, the kit also includes: bisulfite, DNA purification agent, DNA extraction agent, PCR amplification agent and/or instruction for use (indicating operation steps of the detection and a result judgment standard).

In the eighth aspect of the disclosure, a method for detecting the methylation profile of a sample in vitro is provided, including: (i) providing the sample and extracting the nucleic acid; (ii) detecting the modification on CPG site(s) of a target sequence in the nucleic acid of (i), wherein the target sequence is the polynucleotide described in the first aspect or the polynucleotide converted therefrom, which described in the second aspect.

In a preferable embodiment, in step (3), the analysis methods include pyrosequencing, bisulfite conversion sequencing, method using methylation chip, qPCR, digital PCR, second generation sequencing, third generation sequencing, whole genome methylation sequencing, DNA enrichment detection, simplified bisulfite sequencing technology, HPLC, MassArray, methylation specific PCR (MSP), or their combination, as well as in vitro detection and in vivo tracer detection for the combined gene group of partial or all of the methylation sites in the sequence shown in SEQ ID NO: 1. In addition, other methylation detection methods and newly developed methylation detection methods in the future can be applied to the disclosure.

In another preferable embodiment, step (ii) includes: (1) treating the product of (i) to convert the unmodified cytosine into uracil; preferably, the modification includes 5-methylation, 5-hydroxymethylation, 5-formylcytosine (5fC) or 5-carboxylcytosine (5-caC); preferably, treating the nucleic acid of step (i) with bisulfite; and (2) analyzing the modification of the target sequence in the nucleic acid treated by (1).

In another preferable embodiment, the abnormal methylation profile is the high level of methylation of C in CPG(s) of the polynucleotide.

In another preferable embodiment, the methylation profile detecting method is not for the purpose of directly obtaining the diagnosis result of a disease, or is not a diagnostic method.

The ninth aspect of the disclosure provides a tumor diagnosis kit, including primer pairs designed based on the sequence described in the first or second aspect of the disclosure, and gene Panels or gene groups containing the sequence, to obtain the characteristics of normal cells and tumor cells through DNA methylation detection.

Other aspects of the disclosure will be apparent to those skilled in the art based on the disclosure herein.

### BRIEF DESCRIPTION OF DRAWINGS

FIG. 1 shows the average methylation value of STAMP-EP1 in the genomic DNA of lung cancer cell line A549, with the corresponding value of the genomic DNA of normal lung fibroblast cell line MRC5 as the control. Dark squares indicate the corresponding sites “methylated”, while light squares indicate the corresponding sites “non-methylated”.

FIG. 2 shows comparison of STAMP-EP1 methylation value (left), the detection specificity and sensitivity (right) between control group and experimental group of 20 pairs of paracancerous-lung cancer samples, with paracancerous samples as lung cancer control group, and lung cancer samples as lung cancer experimental group.

FIG. 3. 10 paracancerous clinical samples of colorectal cancer were used as the control group, and 28 colorectal cancer clinical samples were used as the experimental group. The STAMP-EP1 methylation value (left), the detection specificity and sensitivity (right) were compared between the control group and the experimental group.

FIG. 4. 10 normal gastric (or gastritis) clinical samples were used as the control group, 10 gastric cancer resection edge clinical samples were used as the experimental group 1, and 20 gastric cancer clinical samples were used as experimental group 2. The STAMP-EP1 methylation value was compared among the three groups.

FIG. 5. 13 paracancerous clinical samples of cervical cancer were used as the control group, and 26 cervical cancer clinical samples were used as the experimental group. The STAMP-EP1 methylation value (left), the detection specificity and sensitivity (right) were compared between the control group and the experimental group.

FIG. 6 shows comparison of STAMP-EP1 methylation value (left), the detection specificity and sensitivity (right) between control group and experimental group of 21 pairs of paracancerous-liver cancer samples, with paracancerous samples as control group, and liver cancer samples as experimental group.

FIG. 7 shows comparison of STAMP-EP1 methylation value (left), the detection specificity and sensitivity (right) between control group and experimental group of 22 pairs of paracancerous-breast cancer samples, with paracancerous samples as control group, and breast cancer samples as experimental group.

FIG. 8 shows comparison of STAMP-EP1 methylation value (left), the detection specificity and sensitivity (right) between control group and experimental group of 18 pairs of paracancerous-pancreatic cancer samples, with paracancerous samples as control group, and pancreatic cancer samples as experimental group.

FIG. 9. Eleven pairs of paracancerous-head and neck cancer samples were obtained, including 5 cases of laryngeal cancer, 2 cases of tonsil cancer, 2 cases of epiglottis cancer, 1 case of tongue base cancer, and 1 case of hypopharyngeal cancer. STAMP-EP1 methylation value was compared between head and neck cancer control group and experimental group, with paracancerous samples as control group, and cancer samples as experimental group.

FIG. 10 shows comparison of STAMP-EP1 methylation value (left), the detection specificity and sensitivity (right) between control group and experimental group of bile samples from 12 non-cancer patients and 10 gallbladder cancer patients, with non-cancer samples as control group, and gallbladder cancer samples as experimental group.

FIG. 11. Ten non-leukemia bone marrow smear samples were used as the control group, and 20 leukemia bone marrow smear samples were used as the experimental group. The STAMP-EP1 methylation value (left), the detection specificity and sensitivity (right) were compared between the control group and experimental group.

FIG. 12. Eight paracancerous clinical samples of renal cancer were used as the control group, and 16 renal cancer clinical samples were used as the experimental group. The STAMP-EP1 methylation value (left), the detection specificity and sensitivity (right) were compared between the control group and experimental group.

FIG. 13. Five cases of bladder cancer paracancerous samples and non-cancer urine samples were used as the control group, and 7 cases of bladder cancer tissue samples and bladder cancer urine samples were used as the experimental group. The STAMP-EP1 methylation value was compared between the control group and the experimental group.

FIG. 14. Twenty normal human plasma samples were used as the control group, and plasma samples from patients with different tumor types were obtained, including 10 cases of liver cancer, 10 cases of pancreatic cancer, 10 cases of lung cancer, 10 cases of colorectal cancer and 10 cases of breast cancer. The STAMP-EP1 methylation value was compared between the control group and the experimental group.

### DETAILED DESCRIPTION

The inventor is committed to the research of tumor markers. After extensive research and screening, the inventor provides a universal DNA methylation tumor marker, STAMP (Specific Tumor Aligned Methylation of Pan-cancer). In normal tissues, STAMP was hypomethylated, while in tumor tissues, it was hypermethylated. It can be used for clinical tumor detection and as the basis of designing tumor diagnostic agents.

### Term

As used herein, “isolated” refers to a material separated from its original environment (if the material is a natural material, the original environment is the natural environment). For example, in living cells, polynucleotides and polypeptides in their natural state are not isolated or purified, but the same polynucleotides or polypeptides will be isolated ones if they are separated from other substances existed in the natural state.

As used herein, “sample” includes substances suitable for DNA methylation detection obtained from any individual or isolated tissue, cell or body fluid (such as plasma). For example, the samples include but are not limited to: tissue samples, paraffin embedded samples, blood samples, pleural effusion samples, and alveolar lavage fluid samples, ascites and lavage fluid samples, bile samples, stool samples, urine samples, saliva samples, cerebrospinal fluid samples, cell smear samples, cervical scraping or brushing samples, tissue and cell biopsy samples.

As used herein, “hypermethylation” refers to high level of methylation, hydroxymethylation, 5-formylcytosine (5fC) or 5-carboxylcytosine (5-caC) of CpG in a gene sequence. For example, in the case of methylation specific PCR (MSP), if the PCR reaction with methylation specific primers has positive PCR results, the DNA (gene) region of interest is in hypermethylation state. For another example, in the case of real-time quantitative methylation specific PCR, hypermethylation can be determined based on statistic difference of the methylation status value as compared with the control sample.

As used herein, the tumors include but are not limited to: hematologic cancers such as leukemia, lymphoma, multiple myeloma; digestive system tumors such as esophageal cancer, gastric cancer, colorectal cancer, liver cancer, pancreatic cancer, bile duct and gallbladder cancer; respiratory system tumors such as lung cancer, pleuroma; nervous system tumors such as glioma, neuroblastoma, meningioma; head and neck tumors such as oral cancer, tongue cancer, laryngeal cancer, nasopharyngeal cancer; gynecological and reproductive system tumors such as breast cancer, ovarian cancer, cervical cancer, vulvar cancer, testicular cancer, prostate cancer, penile cancer; urinary system tumors such as kidney cancer, bladder cancer, skin and other systems tumors such as skin cancer, melanoma, osteosarcoma, liposarcoma, thyroid cancer.

Gene Marker

In order to find a useful target for tumor diagnosis, the inventor has identified the target of STAMP-EP1 after extensive and in-depth research. The methylation status of the sequence of STAMP-EP1 gene is significantly different between tumor tissues and non-tumor tissues. If the abnormal hypermethylation of the sequence of STAMP-EP1 gene of a subject is detected, the subject can be identified as having a high-risk of tumor. Moreover, the significant difference of STAMP-EP1 between tumor and non-tumor tissues exists in different types of tumors, including solid tumors and non-solid tumors.

Therefore, the disclosure provides an isolated polynucleotide, comprising the nucleotide sequence shown in the sequence of SEQ ID NO: 1 or SEQ ID NO: 3 (the reverse complementary sequence of SEQ ID NO: 1). For tumor cells, the polynucleotide contains 5-methylcytosine (5mC) at C positions of many 5′-CpG-3′. The disclosure also comprises fragments of the polynucleotide of the sequence shown in SEQ ID NO: 1 or 3, having at least one (such as 2-178, more specifically 3, 5, 10, 15, 20, 25, 30, 50, 60, 80, 100, 120, 150) methylated CpG site. The above polynucleotides or fragments can also be used in the design of detection agents or detection kits.

In some specific embodiments of the disclosure, the fragments of the polynucleotide are, for example, a fragment containing the residues 1-589 of SEQ ID NO: 1 (containing CpG sites 001-059); a fragment containing the residues 632-1218 of SEQ ID NO: 1 (containing CpG sites 060-100); a fragment containing the residues 1322-2066 of SEQ ID NO: 1 (containing CpG sites 101-147); a fragment containing the residues 2100-2448 of SEQ ID NO: 1 (containing CpG sites 148-178). Antisense chains of the above fragments are suitable for use in the disclosure. These fragments are merely examples of preferable embodiments of the present disclosure. Based on the information provided by the present disclosure, other fragments can also be selected.

In addition, gene Panels or gene groups containing the sequence shown in the SEQ ID NO: 1 or SEQ ID NO: 2 or fragments thereof are also encompassed by the disclosure. For the gene Panel or gene group, the characteristics of normal cells and tumor cells can also be identified through DNA methylation detection.

The above polynucleotides can be used as the key regions for analysis of the methylation status in the genome. Their methylation status can be analyzed by various technologies known in the art. Any technique that can be used to analyze the methylation state can be applied to the present disclosure.

When treated with bisulfite, un-methylated cytosine(s) of the above polynucleotides will be converted into uracil, while methylated cytosine(s) remained unchanged.

Therefore, the disclosure also provides the polynucleotides obtained from the above polynucleotides (including the complementary chain (antisense chain)) after being treated with bisulfite, including the polynucleotides of the sequence shown in SEQ ID NO: 2 or SEQ ID NO: 4. These polynucleotides can also be used in the design of detection agents or detection kits.

The disclosure also comprises fragments of the polynucleotides obtained from the above polynucleotides or the antisense chain thereof after being treated with bisulfite, wherein the fragments contain at least one methylated CpG site.

Detection Agents and Kits

Based on the new discovery of the disclosure, a detection agent designed based on said polynucleotide(s) is also provided for detecting the methylation profile of polynucleotide(s) in the sample in vitro. The detection methods and agents known in the art for determining the sequence, variation and methylation of the genome can be applied in the disclosure.

Therefore, the disclosure provides a method of preparing a tumor detection agent, including: providing the polynucleotide, designing a detection agent for specifically detecting a target sequence which is the full length or fragment of the polynucleotide; wherein, the target sequence has at least one methylated CpG site.

The detection agent herein includes but is not limited to: primers, probes, etc.

For example, the agent is primer pairs. Based on the sequence of the polynucleotide, those skilled in the art know how to design primer(s). The two primers are on each side of the specific sequence of the target gene to be amplified (including CpG sequence, for the gene region originally methylated, the primer is complementary with CpG, and for the gene region originally un-methylated, the primer is complementary with TpG). It should be understood that based on the new discovery of the disclosure, those skilled in the art can design a variety of primers or probes or other types of detection agents for CpG sites at different positions on the target sequence or their combinations. These primers or probes or other types of detection agents should be included in the technical solution of the present disclosure.

The agent can also be a combination of agents (primer combination), including more than one set of primers, so that the multiple polynucleotides can be amplified respectively.

The disclosure also provides a kit for detecting the methylation profile of polynucleotide in a sample in vitro, which comprises container(s) and the above primer pair(s) in the container(s).

In addition, the kit can also include various reagents required for DNA extraction, DNA purification, PCR amplification, etc.

In addition, the kit can also include an instruction for use, which indicates operation steps of the detection and a result judgment standard, for the application of those skilled in the art.

Detection Method

The methylation profile of a polynucleotide can be determined by any technique in the art (such as methylation specific PCR (MSP) or real-time quantitative methylation specific PCR, Methylight), or other techniques that are still developing and will be developed.

Quantitative methylation specific PCR (QMSP) can also be used to detect methylation level. It is a continuous optical monitoring method based on fluorescent PCR, which is more sensitive than MSP. It has high throughput and avoids electrophoresis based result analysis.

Other available technologies include conventional methods in the art such as pyrosequencing, bisulfite converstion sequencing, qPCR, second generation sequencing, whole genome methylation sequencing, DNA enrichment detection, simplified bisulfite sequencing or HPLC, and combined gene group detection. It should be understood that, on the basis of the new disclosure herein, these well-known technologies and some technologies to be developed in the art can be applied to the present disclosure.

As a preferable embodiment of the disclosure, a method of detecting the methylation profile of a polynucleotide in a sample in vitro is also provided. The method is based on the follow principle: the un-methylated cytosine can be converted into uracil by bisulfite, which can be transformed into thymine in the subsequent PCR amplification process, while the methylated cytosine remains unchanged; therefore, after the polynucleotide is treated by bisulfite, the methylated site presents a polynucleotide polymorphism (SNP) similar to C/T. Based on the above principle, methylated and un-methylated cytosine can be distinguished effectively by identifying the methylation profile of a polynucleotide in the sample.

The method of the disclosure includes: (a) providing samples and extracting genomic DNA; (b) treating the genomic DNA of step (a) with bisulfite, so as to convert the un-methylated cytosine in the genomic DNA into uracil; (c) analyzing whether the genomic DNA treated in step (b) contains an abnormal methylation profile.

The method of the disclosure can be used for: (i) analyzing whether a subject has tumor by detecting the sample of the subject; (ii) identifying a population having high-risk of tumor. The method needs not to be aimed at obtaining direct diagnosis results.

In a preferable embodiment of the disclosure, DNA methylation is detected by PCR amplification and pyrosequencing. It should be understood by those in the art that DNA methylation detection is not limited to these methods, and any other DNA methylation detection method can also be used. The primers used in PCR amplification are not limited to those provided in Examples.

Because of bisulfite treatment, in which un-methylated cytosine in genomic DNA are converted into uracil and then transformed into thymine in the subsequent PCR process, the sequence complexity of the genome will be reduced, and it will be more difficult to amplify specific target fragments by PCR. Therefore, in order to improve amplification efficiency and specificity, nested PCR may be preferable, wherein two sets of primers (outer primers and inner primers) are used in two successive runs of PCR, and the amplification product from the first run undergoes a second run with the second set of primers. However, it should be understood that the detection methods suitable for the present disclosure are not limited thereto.

After the research and verification on clinical samples, the method of the disclosure provides very high accuracy in the clinical diagnosis of tumors. The disclosure can be applied to the fields of tumor auxiliary diagnosis, efficacy evaluation, prognosis monitoring, etc., thus has a high clinical value.

The disclosure is further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3rd ed. Science Press, 2002) or followed the manufacturer's recommendation.

### Example 1. Nucleic Acid Sequence for STAMP-EP1 Detection

The sequence of the STAMP-EP1 tumor marker is provided as follows: SEQ ID NO: 1 (chr14: 60975733 bp˜60978180 bp (hg19/Human)), in which the underlined bases are methylated CpG sites, and each number below the underline indicates the site number.

| 1~50 bp | CGGCGGGCGC TGTCGAGCAC GGGGAGGTGC TGAAATAGTC CTGGCGTGCT |
|---|---|
| | 001 002 003 004 005 006 |
| 51~100 bp | GATTCAAGCT TTGATTGGCA GAGCCACCCG GTGACTGACA GGGGGTCTCC |
| | 007 |
| 101~150 bp | ATGGCGCCCG CGCCGCCAAT CCGCCCACCC CAATAGCGGA GCCAGCTCGC |
| | 008 009 010 011 012 013 014 |
| 151~200 bp | CTGCCGGCGT GCCTGAGCCG AGCCGAGCCC GAACCCCAAG CCGCGGAGCC |
| | 015 016 017 018 019 020 021 |
| 201~250 bp | AGCACCTCCT CCAGTCGGGG TCGTCCGCTC CCGGCCGTTG AGCCACCGCC |
| | 022 023 024 025 026 027 028 |
| 251~300 bp | GCCACCCGGT AGTGTGTCCC GCTGCCCCAA TCCGCCTCAT CAACAAGCGC |
| | 029 030 031 032 |
| 301~350 bp | CTGGCACACT CAGCCAGGCC CGCGGGCATC TGCTGCGTGT CCCGCTCCGG |
| | 033 034 035 036 037 |
| 351~400 bp | GCTCAGTGCC CTCGCCGCCG CCGGCACTGC CTCGATGTTC CAGCTGCCCA |
| | 038 039 040 041 042 |
| 401~450 bp | TCTTGAATTT CAGCCCCCAG CAAGTGGCCG GGGTATGTGA GACCCTGGAA |
| | 043 |
| 451~500 bp | GAGAGCGGCG ATGTGGAGCG CCTGGGTCGC TTCCTCTGGT CGCTGCCCGT |
| | 044 045 046 047 048 049 |
| 501~550 bp | GGCCCCTGCG GCCTGCGAGG CCCTCAACAA GAATGAGTCG GTGCTACGCG |
| | 050 051 052 053 054 |
| 551~600 bp | CACGAGCCAT CGTGGCCTTT CACGGTGGCA ACTACCGCGA GCTCTATCAT |
| | 055 056 057 058 059 |
| 601~650 bp | ATCCTGGAAA ACCACAAGTT CACCAAGGAG TCGCACGCCA AGCTGCAGGC |
| | 060 061 062 |
| 651~700 bp | GCTGTGGCTT GAAGCACACT ACCAGGAGGC TGAGAAGCTG CGTGGAAGAC |
| | 063 |
| 701~750 bp | 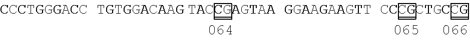 |
| 751~800 bp | 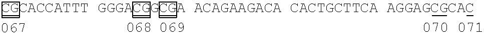 |
| 801~850 bp | GCGGCACCTG CTACGCGAGT GGTACCTGCA GGATCCATAC CCTAACCCCA |
| | 072 073 074 |
| 851~900 bp | GCAAAAAACG TGAGCTCGCC CAGGCAACCG GACTGACCCC TACGCAGGTG |
| | 075 076 077 078 |
| 901~950 bp | GGCAACTGGT TCAAAAACCG CCGACAAAGG GACCGAGCGG CTGCAGCCAA |
| | 079 080 081 082 |
| 951~1000 bp | GAACAGGTCG GTACCTAGAG GCCTCCGCGC TTTGAGCGCA CCGGGGAGGA |
| | 083 084 085 086 087 |
| 1001~1050 bp | GGCGGGTGGA GGCACCTCTG GCGCCCTTAC CCAGTCCCTG GCGACTCCAA |
| | 088 089 090 |
| 1051~1100 bp | TTCAGCAGGA GTTGGGAGCG CGGTCTGTCT TGGGTTAAGA GCCCTGCGTT |
| | 091 092 093 |
| 1101~1150 bp | CTGGGCTCCT GGCCGGGAGT TCCCTTGCCG GCTCTGCTTC CCCACCCGCT |
| | 094 095 096 |
| 1151~1200 bp | GGCTCCCCAC GCCTGCGGGC AGCTGCAGCA GCTGGTCCCG GTCACCAAAC |
| | 097 098 099 |
| 1201~1250 bp | CAAGGCTTCA CTGGGACGGA GAGGGGAAGA GAAATAAAAA ATTAAAATCC |
| | 100 |
| 1251~1300 bp | TACAAACAGT TAGGGACCCC AAGACCCAAA GCTAATTCTT GTCAGCCTGG |
| 1301~1350 bp | GCACAGGCTC CTACTATTAA TCGAAGCCTG GCTTATTAGC AATGTGTCGG |
| | 101 102 |
| 1351~1400 bp | TTTCATGTTA ATTATCATTT TCAAAGCCCA GGTATATCCC TCCCTAATGC |
| 1401~1450 bp | TTTGAAAACA GTTTTCAATG GACTTTTGAG AAATGGGAAG TCGAGTTTTC |
| | 103 |
| 1451~1500 bp | CTCTTCCCAT GCGCTGCCTG CCACTCTTGT CTCAAAACAG CAAACTAGTC |
| | 104 |
| 1501~1550 bp | CGTGGGCCGA GGCTTTTCGT TTCCCGGAGT GTGGATCTCG ATTAGCCAAA |
| | 105 106 107 108 109 |
| 1551~1600 bp | CATTTTGCGG AAGAGCCCGG CCTCATCCCC CAGGCCCAAA TGCTCCTTAC |
| | 110 111 |
| 1601~1650 bp | AATCCTTTTT GCCTTTAGGT CGGGCCGACC CGATCCAACG CGATCGCGGG |
| | 112 113 114 115 116 117 118 |
| 1651~1700 bp | AGCACTTGCT CAGGCGTAAG CCCCAGGCAG ACGCACCGTT AGAAATGGTA |
| | 119 120 121 |
| 1701~1750 bp | TCCCATGTCC CTGGGACCGA TCTGTCCTTG TCACCCACAC TTCGTTTATT |
| | 122 123 |
| 1751~1800 bp | TCCTGACAGT CCTGTAAATC TCCCAAAAGT GCACAACAAA CAGGGAGGAC |
| 1801~1850 bp | ACTGCAAGCC CAGTATATAA AAGACCTGGG AGCTGCGGCG CTGAGAAAGG |
| | 124 125 |
| 1851~1900 bp | GCGCGAATCA TGGTGGGGCA CAACAGTAGG GACCCGCGGA GGGGCGGCCG |
| | 126 127 128 129 130 131 |
| 1901~1950 bp | CGGACTCCTG CCCGACCTCT GTCGCCTTGC CGAGTAATCC TCGCCTTAAC |
| | 132 133 134 135 136 |
| 1951~2000 bp | TGCTGGGGTC TTCGGAAGAA CCTCTAGCCG CCGGGCTGGA GGGACGCAGG |
| | 137 138 139 140 |
| 2001~2050 bp | AGGTGGTGGG GGCGGGCGAC GGGCGGCTGT GTTACGAGCT GTGACCCGTG |
| | 141 142 143 144 145 146 |
| 2051~2100 bp | TTCCCTTTCT TCCCCGTAGA CTCCAGCAGC AGGTCCTGTC ACAGGGTTCC |
| | 147 148 |
| 2101~2150 bp | GGGCGGGCAC TACGGGCGGA GGGCGACGGC ACGCCAGAGG TGCTGGGCGT |
| | 149 150 151 152 153 154 155 |
| 2151~2200 bp | CGCCACCAGC CCGGCCGCCA GTCTATCCAG CAAGGCGGCC ACTTCAGCCA |
| | 156 157 158 159 |
| 2201~2250 bp | TCTCCATCAC GTCCAGCGAC AGCGAGTGCG ACATCTGAGT TGCCCATCCA |
| | 160 161 162 163 |
| 2251~2300 bp | GGATGCTCAG AAGCAGATTC CAGTGTAAAA ACGAGAAAAA CAAAATGAAA |
| | 164 |
| 2301~2350 bp | GAGGGGAAGA AGATGAGAGA CCTGCAAATC CAGCGCCACA GAAGCCAGGT |
| | 165 |
| 2351~2400 bp | GACCAGGGAC CCGCGGGCTC GGGTTGCCGT TTCCCGCCCC ACCCCGCGGC |
| | 166 167 168 169 170 171 172 |
| 2401~2448 bp | CGGCCTGGCT TCACTGGCGC CCTTTGGCCG CGACCACGGG AACCAGCG |
| | 173 174 175 176 177 178 |

The bisulfite treated sequence from SEQ ID NO: 1 is shown in SEQ ID NO: 2 (Y represents C or U) as follows:

| 1~50 bp | YGGYGGGYGU TGTYGAGUAY GGGGAGGTGU TGAAATAGTU UTGGYGTGUT |
|---|---|
| | 001 002003 004 005 006 |
| 51~100 bp | GATTUAAGUT TTGATTGGUA GAGUUAUUYG GTGAUTGAUA GGGGGTUTUU |
| | 007 |
| 101~150 bp | ATGGYGUUYG YGUYGUUAAT UYGUUUAUUU UAATAGYGGA GUUAGUTYGU |
| | 008 009 010 011 012 013 014 |
| 151~200 bp | UTGUYGGYGT GUUTGAGUYG AGUYGAGUUY GAAUUUUAAG UYGYGGAGUU |
| | 015 016 017 018 019 020 021 |
| 201~250 bp | AGUAUUTUUT UUAGTYGGGG TYGTUYGUTU UYGGUYGTTG AGUUAUYGUY |
| | 022 023 024 025 026 027 028 |
| 251~300 bp | GUUAUUYGGT AGTGTGTUUY GUTGUUUUAA TUYGUUTUAT UAAUAAGYGU |
| | 029 030 031 032 |
| 301~350 bp | UTGGUAUAUT UAGUUAGGUU YGYGGGUATU TGUTGYGTGT UUYGUTUYGG |
| | 033 034 035 036 037 |
| 351~400 bp | GUTUAGTGUU UTYGUYGUYG UYGGUAUTGU UTYGATGTTU UAGUTGUUUA |
| | 038 039040 041 042 |
| 401~450 bp | TUTTGAATTT UAGUUUUUAG UAAGTGGUYG GGGTATGTGA GAUUUTGGAA |
| | 043 |
| 451~500 bp | GAGAGYGGYG ATGTGGAGYG UUTGGGTYGU TTUUTUTGGT YGUTGUUYGT |
| | 044045 046 047 048 049 |
| 501~550 bp | GGUUUUTGYG GUUTGYGAGG UUUTUAAUAA GAATGAGTYG GTGUTAYGYG |
| | 050 051 052 053 054 |
| 551~600 bp | UAYGAGUUAT YGTGGUUTTT UAYGGTGGUA AUTAUYGYGA GUTUTATUAT |
| | 055 056 057 058 059 |
| 601~650 bp | ATUUTGGAAA AUUAUAAGTT UAUUAAGGAG TYGUAYGUUA AGUTGUAGGY |
| | 060 061 062 |
| 651~700 bp | GUTGTGGUTT GAAGUAUAUT AUUAGGAGGU TGAGAAGUTG YGTGGAAGAU |
| | 063 |
| 701~750 bp | UUUTGGGAUU TGTGGAUAAG TAUYGAGTAA GGAAGAAGTT UUYGUTGUYG |
| | 064 065 066 |
| 751~800 bp | YGUAUUATTT GGGAYGGYGA AUAGAAGAUA UAUTGUTTUA AGGAGYGUAY |
| | 067 068 069 070 071 |
| 801~850 bp | GYGGUAUUTG UTAYGYGAGT GGTAUUTGUA GGATUUATAU UUTAAUUUUA |
| | 072 073 074 |
| 851~900 bp | GUAAAAAAYG TGAGUTYGUU UAGGUAAUYG GAUTGAUUUU TAYGUAGGTG |
| | 075 076 077 078 |
| 901~950 bp | GGUAAUTGGT TUAAAAAUYG UYGAUAAAGG GAUYGAGYGG UTGUAGUUAA |
| | 079 080 081 082 |
| 951~1000 bp | GAAUAGGTYG GTAUUTAGAG GUUTUYGYGU TTTGAGYGUA UYGGGGAGGA |
| | 083 084 085 086 087 |
| 1001~1050 bp | GGYGGGTGGA GGUAUUTUTG GYGUUUTTAU UUAGTUUUTG GYGAUTUUAA |
| | 088 089 090 |
| 1051~1100 bp | TTUAGUAGGA GTTGGGAGYG YGGTUTGTUT TGGGTTAAGA GUUUTGYGTT |
| | 091 092 093 |
| 1101~1150 bp | UTGGGUTUUT GGUYGGGAGT TUUUTTGUYG GUTUTGUTTU UUUAUUYGUT |
| | 094 095 096 |
| 1151~1200 bp | GGUTUUUUAY GUUTGYGGGU AGUTGUAGUA GUTGGTUUYG GTUAUUAAAU |
| | 097 098 099 |
| 1201~1250 bp | UAAGGUTTUA UTGGGAYGGA GAGGGGAAGA GAAATAAAAA ATTAAAATUU |
| | 100 |
| 1251~1300 bp | TAUAAAUAGT TAGGGAUUUU AAGAUUUAAA GUTAATTUTT GTUAGUUTGG |
| 1301~1350 bp | GUAUAGGUTU UTAUTATTAA TYGAAGUUTG GUTTATTAGU AATGTGTYGG |
| | 101 102 |
| 1351~1400 bp | TTTUATGTTA ATTATUATTT TUAAAGUUUA GGTATATUUU TUUUTAATGU |
| 1401~1450 bp | TTTGAAAAUA GTTTTUAATG GAUTTTTGAG AAATGGGAAG TYGAGTTTTU |
| | 103 |
| 1451~1500 bp | UTUTTUUUAT GYGUTGUUTG UUAUTUTTGT UTUAAAAUAG UAAAUTAGTU |
| | 104 |
| 1501~1550 bp | YGTGGGUYGA GGUTTTTYGT TTUUYGGAGT GTGGATUTYG ATTAGUUAAA |
| | 105 106 107 108 109 |
| 1551~1600 bp | UATTTTGYGG AAGAGUUYGG UUTUATUUUU UAGGUUUAAA TGUTUUTTAU |
| | 110 111 |
| 1601~1650 bp | AATUUTTTTT GUUTTTAGGT YGGGUYGAUU YGATUUAAYG YGATYGYGGG |
| | 112 113 114 115 116 117118 |
| 1651~1700 bp | AGUAUTTGUT UAGGYGTAAG UUUUAGGUAG AYGUAUYGTT AGAAATGGTA |
| | 119 120 121 |
| 1701~1750 bp | TUUUATGTUU UTGGGAUYGA TUTGTUUTTG TUAUUUAUAU TTYGTTTATT |
| | 122 123 |
| 1751~1800 bp | TUUTGAUAGT UUTGTAAATU TUUUAAAAGT GUAUAAUAAA UAGGGAGGAU |
| 1801~1850 bp | AUTGUAAGUU UAGTATATAA AAGAUUTGGG AGUTGYGGYG UTGAGAAAGG |
| | 124125 |
| 1851~1900 bp | GYGYGAATUA TGGTGGGGUA UAAUAGTAGG GAUUYGYGGA GGGGYGGUYG |
| | 126127 128129 130131 |
| 1901~1950 bp | YGGAUTUUTG UUYGAUUTUT GTYGUUTTGU YGAGTAATUU TYGUUTTAAU |
| | 132 133 134 135 136 |
| 1951~2000 bp | TGUTGGGGTU TTYGGAAGAA UUTUTAGUYG UYGGGUTGGA GGGAYGUAGG |
| | 137 138 139 140 |
| 2001~2050 bp | AGGTGGTGGG GGYGGGYGAY GGGYGGUTGT GTTAYGAGUT GTGAUUYGTG |
| | 141 142143 144 145 146 |
| 2051~2100 bp | TTUUUTTTUT TUUUYGTAGA UTUUAGUAGU AGGTUUTGTU AUAGGGTTUY |
| | 147 148 |
| 2101~2150 bp | GGGYGGGUAU TAYGGGYGGA GGGYGAYGGU AYGUUAGAGG TGUTGGGYGT |
| | 149 150 151 152 153 154 155 |
| 2151~2200 bp | YGUUAUUAGU UYGGUYGUUA GTUTATUUAG UAAGGYGGUU AUTTUAGUUA |
| | 156 157 158 159 |
| 2201~2250 bp | TUTUUATUAY GTUUAGYGAU AGYGAGTGYG AUATUTGAGT TGUUUATUUA |
| | 160 161 162 163 |
| 2251~2300 bp | GGATGUTUAG AAGUAGATTU UAGTGTAAAA AYGAGAAAAA UAAAATGAAA |
| | 164 |
| 2301~2350 bp | GAGGGGAAGA AGATGAGAGA UUTGUAAATU UAGYGUUAUA GAAGUUAGGT |
| | 165 |
| 2351~2400 bp | GAUUAGGGAU UYGYGGGUTY GGGTTGUYGT TTUUYGUUUU AUUUYGYGGU |
| | 166167 168 169 170 171 172 |
| 2401~2448 bp | YGGUUTGGUT TUAUTGGYGU UUTTTGGUYG YGAUUAYGGG AAUUAGYG |
| | 173 174 175 176 177 178 |

The reverse complementary sequence of SEQ ID NO: 1 is shown in SEQ ID NO: 3 as follows:

CGCTGGTTCCCGTGGTCGCGGCCAAAGGGCGCCAGTGAAGCCAGGCCGGCC
GCGGGGTGGGGCGGGAAACGGCAACCCGAGCCCGCGGGTCCCTGGTCACCT
GGCTTCTGTGGCGCTGGATTTGCAGGTCTCTCATCTTCTTCCCCTCTTTCA
TTTTGTTTTTCTCGTTTTTACACTGGAATCTGCTTCTGAGCATCCTGGATG
GGCAACTCAGATGTCGCACTCGCTGTCGCTGGACGTGATGGAGATGGCTGA
AGTGGCCGCCTTGCTGGATAGACTGGCGGCCGGGCTGGTGGCGACGCCCAG
CACCTCTGGCGTGCCGTCGCCCTCCGCCCGTAGTGCCCGCCCGGAACCCTG
TGACAGGACCTGCTGCTGGAGTCTACGGGGAAGAAAGGGAACACGGGTCAC
AGCTCGTAACACAGCCGCCCGTCGCCCGCCCCCACCACCTCCTGCGTCCCT
CCAGCCCGGCGGCTAGAGGTTCTTCCGAAGACCCCAGCAGTTAAGGCGAGG
ATTACTCGGCAAGGCGACAGAGGTCGGGCAGGAGTCCGCGGCCGCCCCTCC
GCGGGTCCCTACTGTTGTGCCCCACCATGATTCGCGCCCTTTCTCAGCGCC
GCAGCTCCCAGGTCTTTTATATACTGGGCTTGCAGTGTCCTCCCTGTTTGT
TGTGCACTTTTGGGAGATTTACAGGACTGTCAGGAAATAAACGAAGTGTGG
GTGACAAGGACAGATCGGTCCCAGGGACATGGGATACCATTTCTAACGGTG
CGTCTGCCTGGGGCTTACGCCTGAGCAAGTGCTCCCGCGATCGCGTTGGAT
CGGGTCGGCCCGACCTAAAGGCAAAAAGGATTGTAAGGAGCATTTGGGCCT
GGGGGATGAGGCCGGGCTCTTCCGCAAAATGTTTGGCTAATCGAGATCCAC
ACTCCGGGAAACGAAAAGCCTCGGCCCACGGACTAGTTTGCTGTTTTGAGA
CAAGAGTGGCAGGCAGCGCATGGGAAGAGGAAAACTCGACTTCCCATTTCT
CAAAAGTCCATTGAAAACTGTTTTCAAAGCATTAGGGAGGGATATACCTGG
GCTTTGAAAATGATAATTAACATGAAACCGACACATTGCTAATAAGCCAGG
CTTCGATTAATAGTAGGAGCCTGTGCCCAGGCTGACAAGAATTAGCTTTGG
GTCTTGGGGTCCCTAACTGTTTGTAGGATTTTAATTTTTTATTTCTCTTCC
CCTCTCCGTCCCAGTGAAGCCTTGGTTTGGTGACCGGGACCAGCTGCTGCA
GCTGCCCGCAGGCGTGGGGAGCCAGCGGGTGGGGAAGCAGAGCCGGCAAGG
GAACTCCCGGCCAGGAGCCCAGAACGCAGGGCTCTTAACCCAAGACAGACC
GCGCTCCCAACTCCTGCTGAATTGGAGTCGCCAGGGACTGGGTAAGGGCGC
CAGAGGTGCCTCCACCCGCCTCCTCCCCGGTGCGCTCAAAGCGCGGAGGCC
TCTAGGTACCGACCTGTTCTTGGCTGCAGCCGCTCGGTCCCTTTGTCGGCG
GTTTTTGAACCAGTTGCCCACCTGCGTAGGGGTCAGTCCGGTTGCCTGGGC
GAGCTCACGTTTTTTGCTGGGGTTAGGGTATGGATCCTGCAGGTACCACTC
GCGTAGCAGGTGCCGCGTGCGCTCCTTGAAGCAGTGTGTCTTCTGTTCGCC
GTCCCAAATGGTGCGCGGCAGCGGGAACTTCTTCCTTACTCGGTACTTGTC
CACAGGTCCCAGGGGTCTTCCACGCAGCTTCTCAGCCTCCTGGTAGTGTGC
TTCAAGCCACAGCGCCTGCAGCTTGGCGTGCGACTCCTTGGTGAACTTGTG
GTTTTCCAGGATATGATAGAGCTCGCGGTAGTTGCCACCGTGAAAGGCCAC
GATGGCTCGTGCGCGTAGCACCGACTCATTCTTGTTGAGGGCCTCGCAGGC
CGCAGGGGCCACGGGCAGCGACCAGAGGAAGCGACCCAGGCGCTCCACATC
GCCGCTCTCTTCCAGGGTCTCACATACCCCGGCCACTTGCTGGGGGCTGAA
ATTCAAGATGGGCAGCTGGAACATCGAGGCAGTGCCGGCGGCGGCGAGGGC
ACTGAGCCCGGAGCGGGACACGCAGCAGATGCCCGCGGGCCTGGCTGAGTG
TGCCAGGCGCTTGTTGATGAGGCGGATTGGGGCAGCGGGACACACTACCGG
GTGGCGGCGGTGGCTCAACGGCCGGGAGCGGACGACCCCGACTGGAGGAGG
TGCTGGCTCCGCGGCTTGGGGTTCGGGCTCGGCTCGGCTCAGGCACGCCGG
CAGGCGAGCTGGCTCCGCTATTGGGGTGGGCGGATTGGCGGCGCGGGCGCC
ATGGAGACCCCCTGTCAGTCACCGGGTGGCTCTGCCAATCAAAGCTTGAAT
CAGCACGCCAGGACTATTTCAGCACCTCCCCGTGCTCGACAGCGCCCGCCG

The bisulfite treated sequence from SEQ ID NO: 3 is shown in SEQ ID NO: 4 (Y represents C or U) as follows:

YGUTGGTTUUYGTGGTYGYGGUUAAAGGGYGUUAGTGAAGUUAGGUYGGUY
GYGGGGTGGGGYGGGAAAYGGUAAUUYGAGUUYGYGGGTUUUTGGTUAUUT
GGUTTUTGTGGYGUTGGATTTGUAGGTUTUTUATUTTUTTUUUUTUTTTUA
TTTTGTTTTTUTYGTTTTTAUAUTGGAATUTGUTTUTGAGUATUUTGGATG
GGUAAUTUAGATGTYGUAUTYGUTGTYGUTGGAYGTGATGGAGATGGUTGA
AGTGGUYGUUTTGUTGGATAGAUTGGYGGUYGGGUTGGTGGYGAYGUUUAG
UAUUTUTGGYGTGUYGTYGUUUTUYGUUYGTAGTGUUYGUUYGGAAUUUTG
TGAUAGGAUUTGUTGUTGGAGTUTAYGGGGAAGAAAGGGAAUAYGGGTUAU
AGUTYGTAAUAUAGUYGUUYGTYGUUYGUUUUUAUUAUUTUUTGYGTUUUT
UUAGUUYGGYGGUTAGAGGTTUTTUYGAAGAUUUUAGUAGTTAAGGYGAGG
ATTAUTYGGUAAGGYGAUAGAGGTYGGGUAGGAGTUYGYGGUYGUUUUTUY
GYGGGTUUUTAUTGTTGTGUUUUAUUATGATTYGYGUUUTTTUTUAGYGUY
GUAGUTUUUAGGTUTTTTATATAUTGGGUTTGUAGTGTUUTUUUTGTTTGT
TGTGUAUTTTTGGGAGATTTAUAGGAUTGTUAGGAAATAAAYGAAGTGTGG
GTGAUAAGGAUAGATYGGTUUUAGGGAUATGGGATAUUATTTUTAAYGGTG
YGTUTGUUTGGGGUTTAYGUUTGAGUAAGTGUTUUYGYGATYGYGTTGGAT
YGGGTYGGUUYGAUUTAAAGGUAAAAAGGATTGTAAGGAGUATTTGGGUUT
GGGGGATGAGGUYGGGUTUTTUYGUAAAATGTTTGGUTAATYGAGATUUAU
AUTUYGGGAAAYGAAAAGUUTYGGUUUAYGGAUTAGTTTGUTGTTTTGAGA
UAAGAGTGGUAGGUAGYGUATGGGAAGAGGAAAAUTYGAUTTUUUATTTUT
UAAAAGTUUATTGAAAAUTGTTTTUAAAGUATTAGGGAGGGATATAUUTGG
GUTTTGAAAATGATAATTAAUATGAAAUYGAUAUATTGUTAATAAGUUAGG
UTTYGATTAATAGTAGGAGUUTGTGUUUAGGUTGAUAAGAATTAGUTTTGG
GTUTTGGGGTUUUTAAUTGTTTGTAGGATTTTAATTTTTTATTTUTUTTUU
UUTUTUYGTUUUAGTGAAGUUTTGGTTTGGTGAUYGGGAUUAGUTGUTGUA
GUTGUUYGUAGGYGTGGGGAGUUAGYGGGTGGGGAAGUAGAGUYGGUAAGG
GAAUTUUYGGUUAGGAGUUUAGAAYGUAGGGUTUTTAAUUUAAGAUAGAUY
GYGUTUUUAAUTUUTGUTGAATTGGAGTYGUUAGGGAUTGGGTAAGGGYGU
UAGAGGTGUUTUUAUUYGUUTUUTUUUYGGTGYGUTUAAAGYGYGGAGGUU
TUTAGGTAUYGAUUTGTTUTTGGUTGUAGUYGUTYGGTUUUTTTGTYGGYG
GTTTTTGAAUUAGTTGUUUAUUTGYGTAGGGGTUAGTUYGGTTGUUTGGGY
GAGUTUAYGTTTTTTGUTGGGGTTAGGGTATGGATUUTGUAGGTAUUAUTY
GYGTAGUAGGTGUYGYGTGYGUTUUTTGAAGUAGTGTGTUTTUTGTTYGUY
GTUUUAAATGGTGYGYGGUAGYGGGAAUTTUTTUUTTAUTYGGTAUTTGTU
UAUAGGTUUUAGGGGTUTTUUAYGUAGUTTUTUAGUUTUUTGGTAGTGTGU
TTUAAGUUAUAGYGUUTGUAGUTTGGYGTGYGAUTUUTTGGTGAAUTTGTG
GTTTTUUAGGATATGATAGAGUTYGYGGTAGTTGUUAUYGTGAAAGGUUAY
GATGGUTYGTGYGYGTAGUAUYGAUTUATTUTTGTTGAGGGUUTYGUAGGU
YGUAGGGGUUAYGGGUAGYGAUUAGAGGAAGYGAUUUAGGYGUTUUAUATY
GUYGUTUTUTTUUAGGGTUTUAUATAUUUYGGUUAUTTGUTGGGGGUTGAA
ATTUAAGATGGGUAGUTGGAAUATYGAGGUAGTGUYGGYGGYGGYGAGGGU
AUTGAGUUYGGAGYGGGAUAYGUAGUAGATGUUYGYGGGUUTGGUTGAGTG
TGUUAGGYGUTTGTTGATGAGGYGGATTGGGGUAGYGGGAUAUAUTAUYGG
GTGGYGGYGGTGGUTUAAYGGUYGGGAGYGGAYGAUUUYGAUTGGAGGAGG
TGUTGGUTUYGYGGUTTGGGGTTYGGGUTYGGUTYGGUTUAGGUAYGUYGG
UAGGYGAGUTGGUTUYGUTATTGGGGTGGGYGGATTGGYGGYGYGGGYGUU
ATGGAGAUUUUUTGTUAGTUAUYGGGTGGUTUTGUUAATUAAAGUTTGAAT
UAGUAYGUUAGGAUTATTTUAGUAUUTUUUYGTGUTYGAUAGYGUUYGUYG

### Example 2. STAMP-EP1: Lung Cancer Cell Line Model Detection-BSP (Bisulfite Sequencing PCR) Detection

1. Genomic DNA was extracted from lung cancer cell line A549 and normal lung fibroblast cell line MRC5;

2. The extracted genomic DNA of A549 and MRC5 cell lines were treated with bisulfite and used as templates for subsequent PCR amplification;

3. Primers were designed according to SEQ ID NO: 1. Four pairs of primers were designed for amplification of different sequence regions. The primer sequences and the detected methylation sites are shown in Table 1.

TABLE 1

| | | Detected |
|---|---|---|
| | | CpG site |
| Primer Name | 5′-3′ Primer Sequence | No. |
| STAMP-EP1-Sanger- | AGGTTTTGTAAAATATTGAAGAAAAGTTTAGGAG | CpG sites |
| 1-Primer-F | (SEQ ID NO: 5) | 001-059 |
| STAMP-EP1-Sanger- | CAACCTCCTAATAATATACTTCAAACCACAA | |
| 1-Primer-R | (SEQ ID NO: 6) | |
| STAMP-EP1-Sanger- | TATTTTGGAAAATTATAAGTTTATTAAGGAG | CpG sites |
| 2-Primer-F | (SEQ ID NO: 7) | 060-100 |
| STAMP-EP1-Sanger- | CAAAAATTAACTTTAAATCTTAAAATCCCTAAC | |
| 2-Primer-R | (SEQ ID NO: 8) | |
| STAMP-EP1-Sanger- | AATTTTTGTTAGTTTGGGTATAGGTTTTTATTAT | CpG sites |
| 3-Primer-F | (SEQ ID NO: 9) | 101-147 |
| STAMP-EP1-Sanger- | ACCCTATAACAAAACCTACTACTAAAATCTAC | |
| 3-Primer-R | (SEQ ID NO: 10) | |
| STAMP-EP1-Sanger- | GAATTATGGTGGGGTATAATAGTAGGGATT | CpG sites |
| 4-Primer-F | (SEQ ID NO: 11) | 148-178 |
| STAMP-EP1-Sanger- | TCTAAACAAACRCTCAACTTACAATTCRAATTC | |
| 4-Primer-R | (SEQ ID NO: 12) | |

4. After PCR amplification, 2% agarose gel electrophoresis was used to detect the specificity of the PCR fragments. The target fragments were recovered by gel cutting and connected to T vector which was transformed into competent Escherichia coli. The bacteria were spread on plate, and clones were selected the next day and sequenced. Ten clones were selected for each fragment for Sanger sequencing.

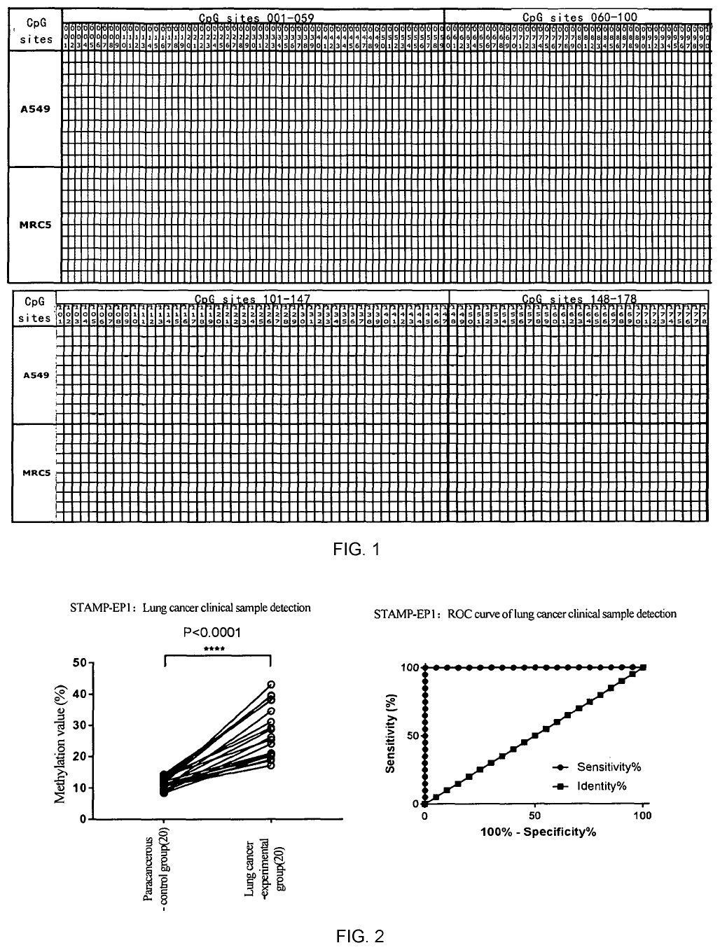

5. The sequencing results are shown in FIG. 1. The average methylation value of lung cancer cell line A549 is 70.73%, while that in normal lung fiber cell line MRC5 is 0.45%.

6. The results showed that STAMP-EP1 was an excellent marker for lung cancer.

### Example 3. STAMP-EP1: Lung Cancer—Clinical Sample Assay—Pyrosequencing

1. Clinical samples: 20 pairs of paracancerous-lung cancer samples were obtained, with paracancerous samples as control group and lung cancer samples as experimental group;

2. DNA extraction: DNA was extracted from the experimental group and the control group respectively. Phenol-chloroform extraction method was used in this experiment, which is not limited thereto;

3. Bisulfite treatment: the extracted DNA samples were treated with bisulfite and the procedures were strictly followed. EZ DNA Methylation-Gold Kit (ZYMO Research, Cat #D5006) was used in this experiment, which is not limited thereto;

4. PCR amplification: the bisulfite treated samples were used as PCR templates, and PCR primers and pyrosequencing primers were designed based on STAMP-EP1 sequences. Following PCR amplification, the methylation values of STAMP-EP1 were detected by pyrosequencing as the methylation values of sites 064, 065, 066, 067, 068 and 069. The PCR primers sequences, pyrosequencing primers sequences, the detecting sequences of pyrosequencing and the detected sites are shown in Table 2;

TABLE 2

| Primer Name | 5′-3′ sequence | Remark |
|---|---|---|
| STAMP-EP1-Pyroseq- | GAAGTATATTATTAGGAGGTTGAGAA | The sites detected |
| Primer-F | G (SEQ ID NO: 13) | by the primer pair: |
| STAMP-EP1-Pyroseq- | TACRCTCCTTAAAACAATATATCTTC | 064, 065, 066, 067, |
| Primer-R(5′- | (SEQ ID NO: 14) | 068, 069 |
| Biotin [symbol] ) | | |
| STAMP-EP1-Pyroseq- | CCCTGGGACCTGTGGACAAGT | |
| Primer-Seq | (SEQ ID NO: 15) | |
| Pyrosequencing | ATYGAGTAAGGAAGAAGTTTTYGTTG | |
| detecting | TYGYGTATTATTTGGGAYGGYG | |
| sequences | (SEQ ID NO: 16) | |

5. Agarose gel electrophoresis: the specificity of PCR amplification was identified by agarose gel electrophoresis, and the size of the amplified fragment should be 139 bp;

6. Pyrosequencing: Pyro Mark Q96 ID pyrosequencing instrument (QIAGEN) was used for sequencing, and the procedures in instructions were strictly followed;

7. Calculation of STAMP-EP1 methylation value: pyrosequencing can detect the methylation values of sites 064, 065, 066, 067, 068, and 069 in the target region, and the average value were calculated as the STAMP-EP1 methylation value in the sample;

8. Results analysis: the methylation value of STAMP-EP1 was compared between the control group and the experimental group, as shown in FIG. 2. It showed that among the clinical samples of lung cancer, the STAMP-EP1 methylation value of the lung cancer experimental group was significantly increased, with P<0.0001, a sensitivity of 100% and a specificity of 100%.

### Example 4. STAMP-EP1: Colorectal Cancer—Clinical Sample Assay—Pyrosequencing

1. Clinical samples: 10 paracancerous clinical samples of colorectal cancer were used as the control group, and 28 colorectal cancer clinical samples were used as the experimental group;

2. Step 2, 3, 4, 5, 6, and 7 are the same as those in Example 3;

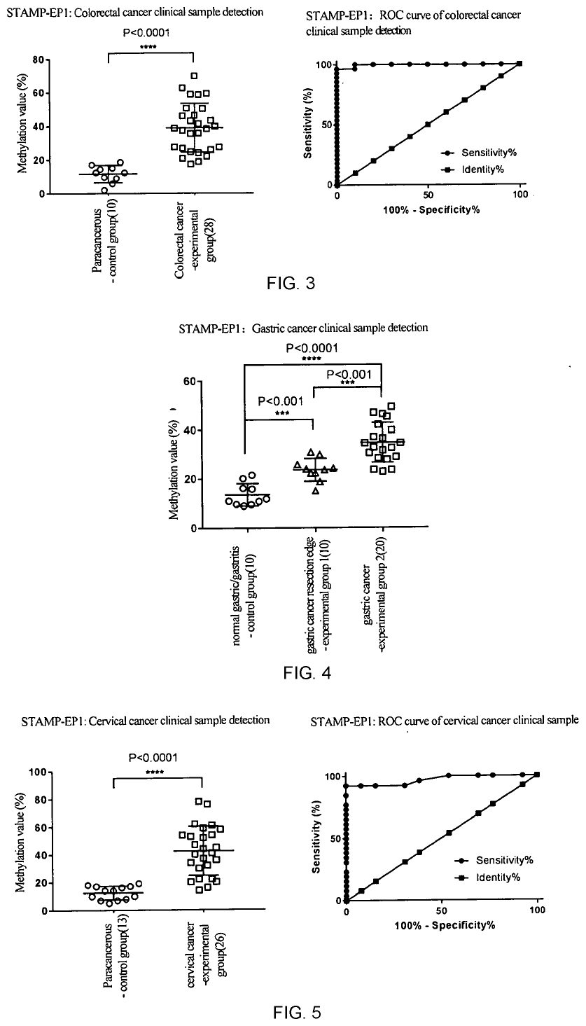

3. Results analysis: the methylation value of STAMP-EP1 was compared between the control group and the experimental group, as shown in FIG. 3. It showed that among the clinical samples of colorectal cancer, the STAMP-EP1 methylation value of the colorectal cancer experimental group was significantly increased, with P<0.0001, a sensitivity of 100% and a specificity of 100%.

### Example 5. STAMP-EP1: Gastric Cancer—Clinical Sample Assay—Pyrosequencing

1. Clinical samples: 10 normal gastric (or gastritis) clinical samples were used as the control group, 10 gastric cancer resection edge clinical samples were used as the experimental group 1, and 20 gastric cancer clinical samples were used as experimental group 2;

2. Step 2, 3, 4, 5, 6, and 7 are the same as those in Example 3; 8. Results analysis: the methylation value of STAMP-EP1 was compared among the normal gastric (or gastritis) control group, the resection edge experimental group 1 and gastric cancer experimental group 2, as shown in FIG. 4. The results showed that among the clinical samples of gastric cancer, the STAMP-EP1 methylation value of the gastric cancer experimental group 2 was significantly increased, P<0.0001. Meanwhile, the methylation of resection edge experimental group 1 was between the normal gastric (or gastritis) control group and the gastric cancer experimental group 2, which indicated that the methylation of STAMP-EP1 began to change at the resection edge. Therefore, as a marker of gastric cancer, STAMP-EP1 can be used to detect gastric cancer, and can also be used to determine the resection edge of gastric cancer.

### Example 6. STAMP-EP1: Cervical Cancer—Clinical Sample Assay—Pyrosequencing

1. Clinical samples: 13 paracancerous clinical samples of cervical cancer were used as the control group, and 26 cervical cancer clinical samples were used as the experimental group;

2. Step 2, 3, 4, 5, 6, and 7 are the same as those in Example 3;

8. Results analysis: the methylation value of STAMP-EP1 was compared between the control group and the experimental group, as shown in FIG. 5. It shows that among the clinical samples of cervical cancer, the STAMP-EP1 methylation value of the cervical cancer experimental group was significantly increased, with P<0.0001, a sensitivity of 92.3% and a specificity of 100%.

### Example 7. STAMP-EP1: Liver Cancer—Clinical Sample Assay—Pyrosequencing

1. Clinical samples: 21 pairs of paracancerous-liver cancer samples were obtained, with paracancerous samples as liver cancer control group and liver cancer samples as liver cancer experimental group;

2. Step 2, 3, 4, 5, 6, and 7 are the same as those in Example 3;

8. Results analysis: the methylation value of STAMP-EP1 was compared between the control group and the experimental group, as shown in FIG. 6. It shows that among the clinical samples of liver cancer, the STAMP-EP1 methylation value of the liver cancer experimental group was significantly increased, with P<0.0001, a sensitivity of 100% and a specificity of 100%.

### Example 8. STAMP-EP1: Breast Cancer—Clinical Sample Assay—Pyrosequencing

1. Clinical samples: 22 pairs of paracancerous-breast cancer samples were obtained, with paracancerous samples as control group and breast cancer samples as experimental group;

2. Step 2, 3, 4, 5, 6, and 7 are the same as those in Example 3;

8. Results analysis: the methylation value of STAMP-EP1 was compared between the control group and the experimental group, as shown in FIG. 7. It shows that among the clinical samples of breast cancer, the STAMP-EP1 methylation value of the breast cancer experimental group was significantly increased, with P<0.0001, a sensitivity of 100% and a specificity of 100%.

### Example 9. STAMP-EP1: Pancreatic Cancer—Clinical Sample Assay—Pyrosequencing

1. Clinical samples: 18 pairs of paracancerous-pancreatic cancer samples were obtained, with paracancerous samples as control group and pancreatic cancer samples as experimental group;

2. Step 2, 3, 4, 5, 6, and 7 are the same as those in Example 3;

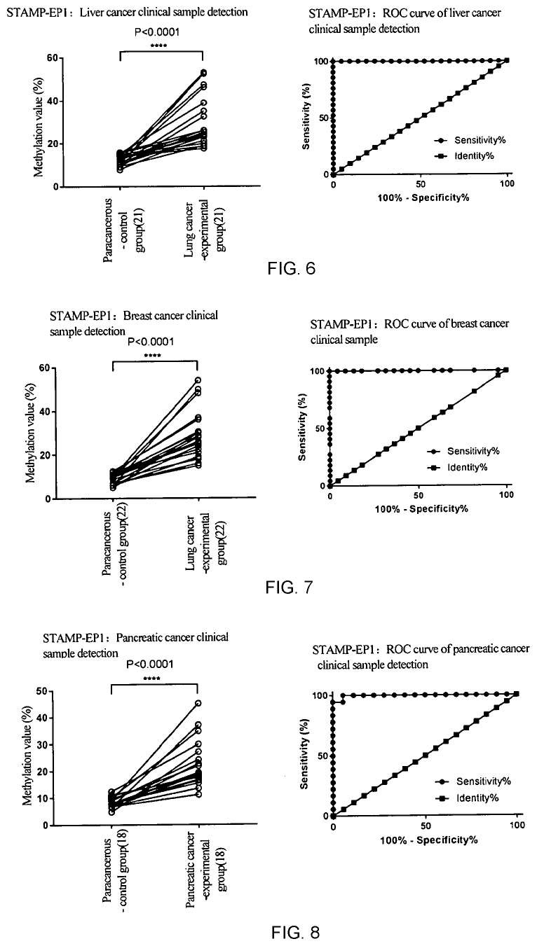

8. Results analysis: the methylation value of STAMP-EP1 was compared between the control group and the experimental group, as shown in FIG. 8. It shows that among the clinical samples of pancreatic cancer, the STAMP-EP1 methylation value of the pancreatic cancer experimental group was significantly increased, with P<0.0001, a sensitivity of 94.44% and a specificity of 100%.

### Example 10. STAMP-EP1: Head and Neck Cancer—Clinical Sample Assay—Pyrosequencing

1. Clinical samples: 11 pairs of paracancerous-head and neck cancer samples were obtained, including 5 cases of laryngeal cancer, 2 cases of tonsil cancer, 2 cases of epiglottis cancer, 1 case of tongue base cancer, and 1 case of hypopharyngeal cancer. Paracancerous samples were used as control group, and cancer samples as experimental group;

2. Step 2, 3, 4, 5, 6, and 7 are the same as those in Example 3;

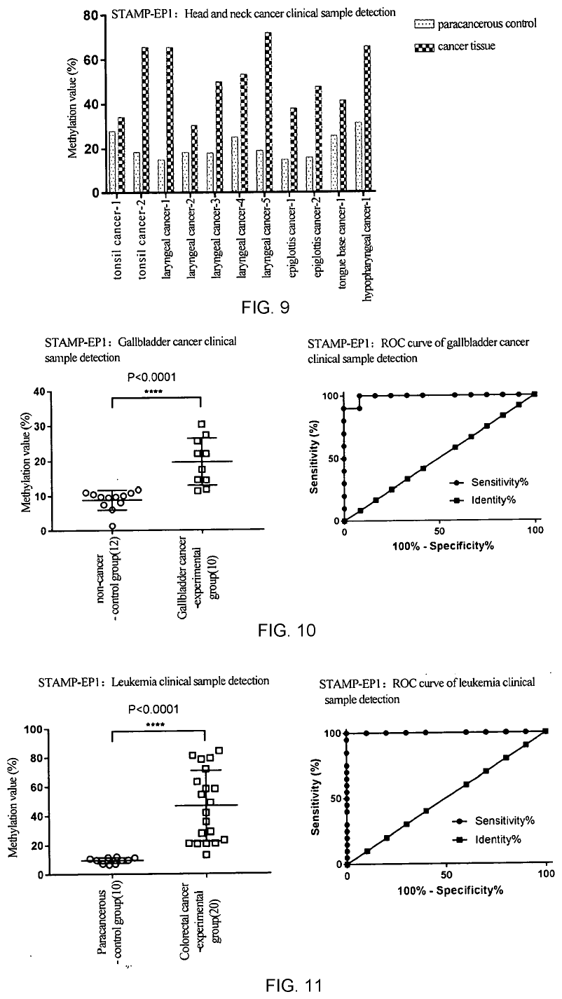

8. Results analysis: the methylation value of STAMP-EP1 was compared between the control group and the experimental group, as shown in FIG. 9. It shows that among the clinical samples of head and neck cancer, the STAMP-EP1 methylation value of the head and neck cancer experimental group was significantly increased, with P<0.0001, a sensitivity of 100% and a specificity of 100%.

### Example 11. STAMP-EP1: Gallbladder Cancer—Clinical Sample Assay—Pyrosequencing

1. Clinical samples: bile samples were obtained from 12 non-cancer patients and 10 gallbladder cancer patients, with non-cancer samples as control group and gallbladder cancer samples as experimental group;

2. Step 2, 3, 4, 5, 6, and 7 are the same as those in Example 3;

8. Results analysis: the methylation value of STAMP-EP1 was compared between the control group and the experimental group, as shown in FIG. 10. It shows that among the clinical samples of gallbladder cancer, the STAMP-EP1 methylation value of the gallbladder cancer experimental group was significantly increased, with P<0.0001, a sensitivity of 90% and a specificity of 100%.

### Example 12. STAMP-EP1: Leukemia—Clinical Sample Assay—Pyrosequencing

1. Clinical samples: 10 non-leukemia bone marrow smear clinical samples were used as the control group, and 20 leukemia bone marrow smear clinical samples were used as the experimental group;

2. Step 2, 3, 4, 5, 6, and 7 are the same as those in Example 3;

8. Results analysis: the methylation value of STAMP-EP1 was compared between the control group and the experimental group, as shown in FIG. 11. It shows that among the clinical samples of leukemia, the STAMP-EP1 methylation value of the leukemia experimental group was significantly increased, with P<0.0001, a sensitivity of 100% and a specificity of 100%.

### Example 13. STAMP-EP1: Renal Cancer—Clinical Sample Assay—Pyrosequencing

1. Clinical samples: 8 paracancerous clinical samples of renal cancer were used as the control group, and 16 renal cancer clinical samples were used as the experimental group;

2. Step 2, 3, 4, 5, 6, and 7 are the same as those in Example 3;

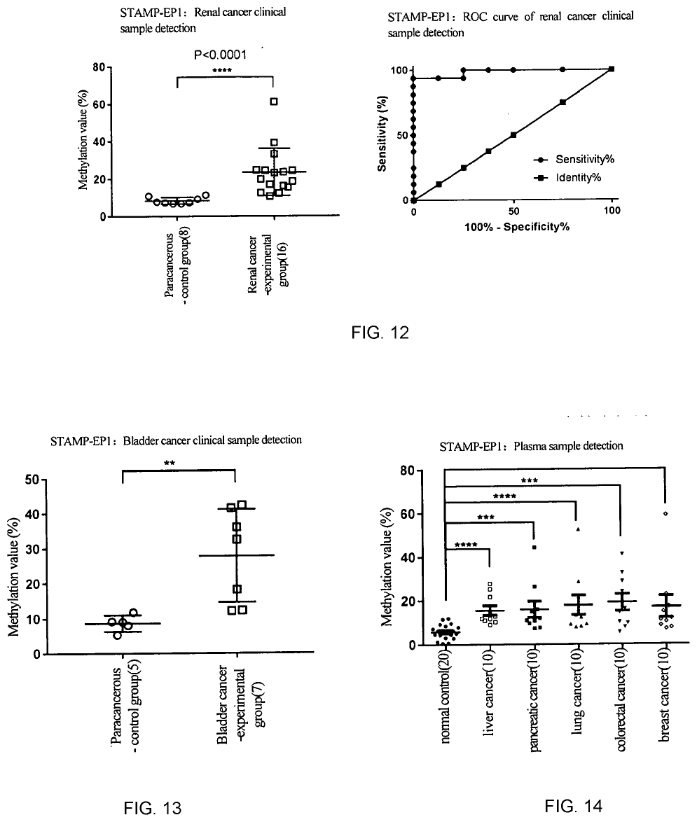

8. Results analysis: the methylation value of STAMP-EP1 was compared between the control group and the experimental group, as shown in FIG. 12. It shows that among the clinical samples of renal cancer, the STAMP-EP1 methylation value of the renal cancer experimental group was significantly increased, with P<0.0001, a sensitivity of 93.75% and a specificity of 100%.

### Example 14. STAMP-EP1: Bladder Cancer—Clinical Sample Assay—Pyrosequencing

1. Clinical samples: 5 cases of bladder cancer paracancerous samples and non-cancer urine samples were used as the control group, and 7 cases of bladder cancer tissue samples and bladder cancer urine samples were used as the experimental group;

2. Step 2, 3, 4, 5, 6, and 7 are the same as those in Example 3;

8. Results analysis: the methylation value of STAMP-EP1 was compared between the control group and the experimental group, as shown in FIG. 13. It shows that among the clinical samples of bladder cancer, the STAMP-EP1 methylation value of the bladder cancer experimental group was significantly increased.

### Example 15. STAMP-EP1: Plasma Sample—Clinical Sample Assay

1. In order to prove that SATMP-C tumor marker can also be detected using liquid biopsy, 20 normal plasma samples were used as the control group, and plasma samples from patients with different tumor types were obtained, including 10 cases of liver cancer, 10 cases of pancreatic cancer, 10 cases of lung cancer, 10 cases of colorectal cancer, and 10 cases of breast cancer;

2. Step 2, 3, 4, 5, 6, and 7 are the same as those in Example 3;

8. Results analysis: as shown in FIG. 14, compared with the normal plasma control group, the methylation values of liver cancer, pancreatic cancer, lung cancer, colorectal cancer and breast cancer groups were significantly increased.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1-16. (canceled)

17. A method for detecting tumor, wherein said method comprising: detecting the modification on the CPG site(s) of a polynucleotide by a tumor detection agent or kit, if the hypermethylation of a subject is detected, the subject can be identified as having a high-risk of tumor; said polynucleotide comprises: (a) the polynucleotide with a nucleotide sequence as shown in SEQ ID NO: 1; (b) a fragment of SEQ ID NO: 1, containing the residues 1-589, the residues 632-1218, the residues 1322-2066 or the residues 2100-2448 of SEQ ID NO: 1; or (c) a nucleic acid complementary to the polynucleotide or fragment of (a) or (b);
wherein the tumors comprise: hematologic cancers such as lymphoma, multiple myeloma; digestive system tumors such as esophageal cancer, gastric cancer, colorectal cancer, liver cancer, pancreatic cancer, bile duct and gallbladder cancer; respiratory system tumors such as pleuroma; nervous system tumors such as glioma, neuroblastoma, meningioma; head and neck tumors such as oral cancer, tongue cancer, laryngeal cancer, nasopharyngeal cancer; gynecological and reproductive system tumors such as breast cancer, ovarian cancer, cervical cancer, vulvar cancer, testicular cancer, prostate cancer, penile cancer; urinary system tumors such as kidney cancer, skin and other systems tumors such as skin cancer, melanoma, osteosarcoma, liposarcoma, thyroid cancer.

18. The method according to claim 17, wherein samples of the tumor comprise: tissue samples, paraffin embedded samples, blood samples, pleural effusion samples, and alveolar lavage fluid samples, ascites and lavage fluid samples, bile samples, stool samples, urine samples, saliva samples, sputum samples, cerebrospinal fluid samples, cell smear samples, cervical scraping or brushing samples, tissue and cell biopsy samples.

19. The method according to claim 17, wherein said tumor detection agent or kit is specifically detect the polynucleotide, or the Panel or gene group containing the polynucleotide.

20. The method according to claim 17, wherein said tumor detection agent or kit comprises primers of probes.

21. The method according to claim 20, wherein said primers are:
the primers shown in SEQ ID NO: 5 and 6;
the primers shown in SEQ ID NO: 7 and 8;
the primers shown in SEQ ID NO: 9 and 10;
the primers shown in SEQ ID NO: 11 and 12; or
the primers shown in SEQ ID NO: 13 and 14.

22. The method according to claim 17, wherein said modification comprises 5-methylation, 5-hydroxymethylation, 5-formylcytosine (5fC) or 5-carboxylcytosine (5-caC).

23. A method of preparing a tumor detection agent, comprising: providing a polynucleotide and designing a detection agent for specifically detecting a target sequence which is the full length or fragment of the polynucleotide; wherein said polynucleotide comprises: (a) the polynucleotide with a nucleotide sequence as shown in SEQ ID NO: 1; (b) a fragment of SEQ ID NO: 1, containing the residues 1-589, the residues 632-1218, the residues 1322-2066 or the residues 2100-2448 of SEQ ID NO: 1; or (c) a nucleic acid complementary to the polynucleotide or fragment of (a) or (b);
wherein the tumors comprise: hematologic cancers such as lymphoma, multiple myeloma; digestive system tumors such as esophageal cancer, gastric cancer, colorectal cancer, liver cancer, pancreatic cancer, bile duct and gallbladder cancer; respiratory system tumors such as pleuroma; nervous system tumors such as glioma, neuroblastoma, meningioma; head and neck tumors such as oral cancer, tongue cancer, laryngeal cancer, nasopharyngeal cancer; gynecological and reproductive system tumors such as breast cancer, ovarian cancer, cervical cancer, vulvar cancer, testicular cancer, prostate cancer, penile cancer; urinary system tumors such as kidney cancer, skin and other systems tumors such as skin cancer, melanoma, osteosarcoma, liposarcoma, thyroid cancer.

24. The method according to claim 23, wherein the detection agent specifically detect a gene sequence containing the target sequence, and the gene sequence comprises a gene Panel or a gene group.

25. The method according to claim 23, wherein the detection agent comprises: primers or probes.

26. The method according to claim 25, wherein the primers are:
the primers shown in SEQ ID NO: 5 and 6;
the primers shown in SEQ ID NO: 7 and 8;
the primers shown in SEQ ID NO: 9 and 10;
the primers shown in SEQ ID NO: 11 and 12; or
the primers shown in SEQ ID NO: 13 and 14.

27. A detection kit, comprising:
container(s) and a detection agent in the container(s), said detection agent comprises primers, the primers are:
the primers shown in SEQ ID NO: 5 and 6;
the primers shown in SEQ ID NO: 7 and 8;
the primers shown in SEQ ID NO: 9 and 10;
the primers shown in SEQ ID NO: 11 and 12; or
the primers shown in SEQ ID NO: 13 and 14.

28. A method of detecting the methylation profile of a sample in vitro, comprising:
(i) providing the sample and extracting nucleic acid;
(ii) detecting the modification on CPG site(s) of a target sequence in the nucleic acid of (i), wherein the target sequence is the polynucleotide comprises: (a) the polynucleotide with a nucleotide sequence as shown in SEQ ID NO: 1; (b) a fragment of SEQ ID NO: 1, containing the residues 1-589, the residues 632-1218, the residues 1322-2066 or the residues 2100-2448 of SEQ ID NO: 1; or (c) a nucleic acid complementary to the polynucleotide or fragment of (a) or (b).

29. The method according to claim 28, wherein, in step (ii), the analysis methods comprise pyrosequencing, bisulfite conversion sequencing, method using methylation chip, qPCR, digital PCR, second generation sequencing, third generation sequencing, whole genome methylation sequencing, DNA enrichment detection, simplified bisulfite sequencing technology, HPLC, MassArray, methylation specific PCR, or their combination, as well as in vitro detection and in vivo tracer detection for the combined gene group of partial or all of the methylation sites in the sequence shown in SEQ ID NO: 1.

30. The method according to claim 28, wherein, step (ii) comprises:
(1) treating the product of (i) to convert the unmodified cytosine into uracil;
(2) analyzing the modification of the target sequence in the nucleic acid treated by (1).

31. The method according to claim 30, wherein treating the nucleic acid of step (i) with bisulfite.

32. An isolated polynucleotide, wherein, the polynucleotide is converted from an original polynucleotide, said original polynucleotide comprises: (a) a fragment of SEQ ID NO: 1, containing the residues 1-589, the residues 632-1218, the residues 1322-2066 or the residues 2100-2448 of SEQ ID NO: 1; or (b) a nucleic acid complementary to the polynucleotide or fragment of (a); and as compared with the sequence of the original polynucleotide, cytosine C of the CpG site(s) with modification is unchanged, and the unmodified cytosine is converted into T or U in the converted polynucleotide.

33. The polynucleotide according to claim 32, wherein treating the nucleic acid of the original polynucleotide with bisulfite to obtain the converted polynucleotide.


SEQUENCE LISTING (16 sequences)

SEQ ID NO: 1 - DNA, 2448 bp, Homo sapiens
  misc_feature (1)..(2448): Wherein Y can be T/U or C
cggcgggcgctgtcgagcacggggaggtgctgaaatagtcctggcgtgctgattcaagctttgattggcagagccacccggtgactgacagggggtctccatggcgcccgcgccgccaatccgcccaccccaatagcggagccagctcgcctgccggcgtgcctgagccgagccgagcccgaaccccaagccgcggagccagcacctcctccagtcggggtcgtccgctcccggccgttgagccaccgccgccacccggtagtgtgtcccgctgccccaatccgcctcatcaacaagcgcctggcacactcagccaggcccgcgggcatctgctgcgtgtcccgctccgggctcagtgccctcgccgccgccggcactgcctcgatgttccagctgcccatcttgaatttcagcccccagcaagtggccggggtatgtgagaccctggaagagagcggcgatgtggagcgcctgggtcgcttcctctggtcgctgcccgtggcccctgcggcctgcgaggccctcaacaagaatgagtcggtgctacgcgcacgagccatcgtggcctttcacggtggcaactaccgcgagctctatcatatcctggaaaaccacaagttcaccaaggagtcgcacgccaagctgcaggcgctgtggcttgaagcacactaccaggaggctgagaagctgcgtggaagacccctgggacctgtggacaagtaccgagtaaggaagaagttcccgctgccgcgcaccatttgggacggcgaacagaagacacactgcttcaaggagcgcacgcggcacctgctacgcgagtggtacctgcaggatccataccctaaccccagcaaaaaacgtgagctcgcccaggcaaccggactgacccctacgcaggtgggcaactggttcaaaaaccgccgacaaagggaccgagcggctgcagccaagaacaggtcggtacctagaggcctccgcgctttgagcgcaccggggaggaggcgggtggaggcacctctggcgcccttacccagtccctggcgactccaattcagcaggagttgggagcgcggtctgtcttgggttaagagccctgcgttctgggctcctggccgggagttcccttgccggctctgcttccccacccgctggctccccacgcctgcgggcagctgcagcagctggtcccggtcaccaaaccaaggcttcactgggacggagaggggaagagaaataaaaaattaaaatcctacaaacagttagggaccccaagacccaaagctaattcttgtcagcctgggcacaggctcctactattaatcgaagcctggcttattagcaatgtgtcggtttcatgttaattatcattttcaaagcccaggtatatccctccctaatgctttgaaaacagttttcaatggacttttgagaaatgggaagtcgagttttcctcttcccatgcgctgcctgccactcttgtctcaaaacagcaaactagtccgtgggccgaggcttttcgtttcccggagtgtggatctcgattagccaaacattttgcggaagagcccggcctcatcccccaggcccaaatgctccttacaatcctttttgcctttaggtcgggccgacccgatccaacgcgatcgcgggagcacttgctcaggcgtaagccccaggcagacgcaccgttagaaatggtatcccatgtccctgggaccgatctgtccttgtcacccacacttcgtttatttcctgacagtcctgtaaatctcccaaaagtgcacaacaaacagggaggacactgcaagcccagtatataaaagacctgggagctgcggcgctgagaaagggcgcgaatcatggtggggcacaacagtagggacccgcggaggggcggccgcggactcctgcccgacctctgtcgccttgccgagtaatcctcgccttaactgctggggtcttcggaagaacctctagccgccgggctggagggacgcaggaggtggtgggggcgggcgacgggcggctgtgttacgagctgtgacccgtgttccctttcttccccgtagactccagcagcaggtcctgtcacagggttccgggcgggcactacgggcggagggcgacggcacgccagaggtgctgggcgtcgccaccagcccggccgccagtctatccagcaaggcggccacttcagccatctccatcacgtccagcgacagcgagtgcgacatctgagttgcccatccaggatgctcagaagcagattccagtgtaaaaacgagaaaaacaaaatgaaagaggggaagaagatgagagacctgcaaatccagcgccacagaagccaggtgaccagggacccgcgggctcgggttgccgtttcccgccccaccccgcggccggcctggcttcactggcgccctttggccgcgaccacgggaaccagcg

SEQ ID NO: 2 - DNA, 2448 bp, Artificial Sequence
  bisulfite treated sequence of seq id no1
  misc_feature (1)..(2448): Wherein Y can be T/U or C
yggygggygutgtygaguayggggaggtgutgaaatagtuutggygtgutgattuaagutttgattgguagaguuauuyggtgautgauagggggtutuuatggyguuygyguyguuaatuyguuuauuuuaatagyggaguuagutyguutguyggygtguutgaguygaguygaguuygaauuuuaaguygyggaguuaguauutuutuuagtyggggtygtuygutuuygguygttgaguuauyguyguuauuyggtagtgtgtuuygutguuuuaatuyguutuatuaauaagyguutgguauautuaguuagguuygyggguatutgutgygtgtuuygutuygggutuagtguuutyguyguyguygguautguutygatgttuuagutguuuatuttgaatttuaguuuuuaguaagtgguyggggtatgtgagauuutggaagagagyggygatgtggagyguutgggtyguttuututggtygutguuygtgguuuutgygguutgygagguuutuaauaagaatgagtyggtgutaygyguaygaguuatygtgguutttuayggtgguaautauygygagututatuatatuutggaaaauuauaagttuauuaaggagtyguayguuaagutguaggygutgtgguttgaaguauautauuaggaggutgagaagutgygtggaagauuuutgggauutgtggauaagtauygagtaaggaagaagttuuygutguygyguauuatttgggayggygaauagaagauauautguttuaaggagyguaygygguauutgutaygygagtggtauutguaggatuuatauuutaauuuuaguaaaaaaygtgagutyguuuagguaauyggautgauuuutayguaggtggguaautggttuaaaaauyguygauaaagggauygagyggutguaguuaagaauaggtyggtauutagagguutuygygutttgagyguauyggggaggaggygggtggagguauututggyguuuttauuuagtuuutggygautuuaattuaguaggagttgggagygyggtutgtuttgggttaagaguuutgygttutgggutuutgguygggagttuuuttguyggututguttuuuuauuygutggutuuuuayguutgyggguagutguaguagutggtuuyggtuauuaaauuaagguttuautgggayggagaggggaagagaaataaaaaattaaaatuutauaaauagttagggauuuuaagauuuaaagutaattuttgtuaguutggguauaggutuutautattaatygaaguutgguttattaguaatgtgtyggtttuatgttaattatuattttuaaaguuuaggtatatuuutuuutaatgutttgaaaauagttttuaatggauttttgagaaatgggaagtygagttttuututtuuuatgygutguutguuaututtgtutuaaaauaguaaautagtuygtggguygagguttttygtttuuyggagtgtggatutygattaguuaaauattttgyggaagaguuygguutuatuuuuuagguuuaaatgutuuttauaatuutttttguutttaggtyggguygauuygatuuaaygygatygygggaguauttgutuaggygtaaguuuuagguagayguauygttagaaatggtatuuuatgtuuutgggauygatutgtuuttgtuauuuauauttygtttatttuutgauagtuutgtaaatutuuuaaaagtguauaauaaauagggaggauautguaaguuuagtatataaaagauutgggagutgyggygutgagaaagggygygaatuatggtgggguauaauagtagggauuygyggaggggygguygyggautuutguuygauututgtyguuttguygagtaatuutyguuttaautgutggggtuttyggaagaauututaguyguygggutggagggayguaggaggtggtgggggygggygaygggyggutgtgttaygagutgtgauuygtgttuuutttuttuuuygtagautuuaguaguaggtuutgtuauagggttuygggyggguautaygggyggagggygaygguayguuagaggtgutgggygtyguuauuaguuygguyguuagtutatuuaguaaggygguuauttuaguuatutuuatuaygtuuagygauagygagtgygauatutgagttguuuatuuaggatgutuagaaguagattuuagtgtaaaaaygagaaaaauaaaatgaaagaggggaagaagatgagagauutguaaatuuagyguuauagaaguuaggtgauuagggauuygygggutygggttguygtttuuyguuuuauuuygygguygguutgguttuautggyguuutttgguygygauuaygggaauuagyg

SEQ ID NO: 3 - DNA, 2448 bp, Homo sapiens
cgctggttcccgtggtcgcggccaaagggcgccagtgaagccaggccggccgcggggtggggcgggaaacggcaacccgagcccgcgggtccctggtcacctggcttctgtggcgctggatttgcaggtctctcatcttcttcccctctttcattttgtttttctcgtttttacactggaatctgcttctgagcatcctggatgggcaactcagatgtcgcactcgctgtcgctggacgtgatggagatggctgaagtggccgccttgctggatagactggcggccgggctggtggcgacgcccagcacctctggcgtgccgtcgccctccgcccgtagtgcccgcccggaaccctgtgacaggacctgctgctggagtctacggggaagaaagggaacacgggtcacagctcgtaacacagccgcccgtcgcccgcccccaccacctcctgcgtccctccagcccggcggctagaggttcttccgaagaccccagcagttaaggcgaggattactcggcaaggcgacagaggtcgggcaggagtccgcggccgcccctccgcgggtccctactgttgtgccccaccatgattcgcgccctttctcagcgccgcagctcccaggtcttttatatactgggcttgcagtgtcctccctgtttgttgtgcacttttgggagatttacaggactgtcaggaaataaacgaagtgtgggtgacaaggacagatcggtcccagggacatgggataccatttctaacggtgcgtctgcctggggcttacgcctgagcaagtgctcccgcgatcgcgttggatcgggtcggcccgacctaaaggcaaaaaggattgtaaggagcatttgggcctgggggatgaggccgggctcttccgcaaaatgtttggctaatcgagatccacactccgggaaacgaaaagcctcggcccacggactagtttgctgttttgagacaagagtggcaggcagcgcatgggaagaggaaaactcgacttcccatttctcaaaagtccattgaaaactgttttcaaagcattagggagggatatacctgggctttgaaaatgataattaacatgaaaccgacacattgctaataagccaggcttcgattaatagtaggagcctgtgcccaggctgacaagaattagctttgggtcttggggtccctaactgtttgtaggattttaattttttatttctcttcccctctccgtcccagtgaagccttggtttggtgaccgggaccagctgctgcagctgcccgcaggcgtggggagccagcgggtggggaagcagagccggcaagggaactcccggccaggagcccagaacgcagggctcttaacccaagacagaccgcgctcccaactcctgctgaattggagtcgccagggactgggtaagggcgccagaggtgcctccacccgcctcctccccggtgcgctcaaagcgcggaggcctctaggtaccgacctgttcttggctgcagccgctcggtccctttgtcggcggtttttgaaccagttgcccacctgcgtaggggtcagtccggttgcctgggcgagctcacgttttttgctggggttagggtatggatcctgcaggtaccactcgcgtagcaggtgccgcgtgcgctccttgaagcagtgtgtcttctgttcgccgtcccaaatggtgcgcggcagcgggaacttcttccttactcggtacttgtccacaggtcccaggggtcttccacgcagcttctcagcctcctggtagtgtgcttcaagccacagcgcctgcagcttggcgtgcgactccttggtgaacttgtggttttccaggatatgatagagctcgcggtagttgccaccgtgaaaggccacgatggctcgtgcgcgtagcaccgactcattcttgttgagggcctcgcaggccgcaggggccacgggcagcgaccagaggaagcgacccaggcgctccacatcgccgctctcttccagggtctcacataccccggccacttgctgggggctgaaattcaagatgggcagctggaacatcgaggcagtgccggcggcggcgagggcactgagcccggagcgggacacgcagcagatgcccgcgggcctggctgagtgtgccaggcgcttgttgatgaggcggattggggcagcgggacacactaccgggtggcggcggtggctcaacggccgggagcggacgaccccgactggaggaggtgctggctccgcggcttggggttcgggctcggctcggctcaggcacgccggcaggcgagctggctccgctattggggtgggcggattggcggcgcgggcgccatggagaccccctgtcagtcaccgggtggctctgccaatcaaagcttgaatcagcacgccaggactatttcagcacctccccgtgctcgacagcgcccgccg

SEQ ID NO: 4 - DNA, 2448 bp, Artificial Sequence
  bisulfite treated sequence of seq id no3
  misc_feature (1)..(2448): y is c or u
  misc_feature (1)..(2448): Wherein Y can be T/U or C
ygutggttuuygtggtygygguuaaagggyguuagtgaaguuagguygguygyggggtggggygggaaaygguaauuygaguuygygggtuuutggtuauutgguttutgtggygutggatttguaggtututuatuttuttuuuututttuattttgtttttutygtttttauautggaatutguttutgaguatuutggatggguaautuagatgtyguautygutgtygutggaygtgatggagatggutgaagtgguyguuttgutggatagautggygguygggutggtggygayguuuaguauututggygtguygtyguuutuyguuygtagtguuyguuyggaauuutgtgauaggauutgutgutggagtutayggggaagaaagggaauaygggtuauagutygtaauauaguyguuygtyguuyguuuuuauuauutuutgygtuuutuuaguuyggyggutagaggttuttuygaagauuuuaguagttaaggygaggattautygguaaggygauagaggtyggguaggagtuygygguyguuuutuygygggtuuutautgttgtguuuuauuatgattygyguuutttutuagyguyguagutuuuaggtuttttatatautggguttguagtgtuutuuutgtttgttgtguauttttgggagatttauaggautgtuaggaaataaaygaagtgtgggtgauaaggauagatyggtuuuagggauatgggatauuatttutaayggtgygtutguutgggguttayguutgaguaagtgutuuygygatygygttggatygggtygguuygauutaaagguaaaaaggattgtaaggaguatttggguutgggggatgagguygggututtuyguaaaatgtttggutaatygagatuuauautuygggaaaygaaaaguutygguuuayggautagtttgutgttttgagauaagagtgguagguagyguatgggaagaggaaaautygauttuuuatttutuaaaagtuuattgaaaautgttttuaaaguattagggagggatatauutgggutttgaaaatgataattaauatgaaauygauauattgutaataaguuagguttygattaatagtaggaguutgtguuuaggutgauaagaattagutttgggtuttggggtuuutaautgtttgtaggattttaattttttatttututtuuuututuygtuuuagtgaaguuttggtttggtgauygggauuagutgutguagutguuyguaggygtggggaguuagygggtggggaaguagaguygguaagggaautuuygguuaggaguuuagaayguagggututtaauuuaagauagauygygutuuuaautuutgutgaattggagtyguuagggautgggtaagggyguuagaggtguutuuauuyguutuutuuuyggtgygutuaaagygyggagguututaggtauygauutgttuttggutguaguygutyggtuuutttgtyggyggtttttgaauuagttguuuauutgygtaggggtuagtuyggttguutgggygagutuaygttttttgutggggttagggtatggatuutguaggtauuautygygtaguaggtguygygtgygutuuttgaaguagtgtgtuttutgttyguygtuuuaaatggtgygygguagygggaauttuttuuttautyggtauttgtuuauaggtuuuaggggtuttuuayguaguttutuaguutuutggtagtgtguttuaaguuauagyguutguaguttggygtgygautuuttggtgaauttgtggttttuuaggatatgatagagutygyggtagttguuauygtgaaagguuaygatggutygtgygygtaguauygautuattuttgttgaggguutyguagguyguagggguuayggguagygauuagaggaagygauuuaggygutuuauatyguygutututtuuagggtutuauatauuuygguuauttgutgggggutgaaattuaagatggguagutggaauatygagguagtguyggyggyggygaggguautgaguuyggagygggauayguaguagatguuygyggguutggutgagtgtguuaggyguttgttgatgaggyggattgggguagygggauauautauygggtggyggyggtggutuaaygguygggagyggaygauuuygautggaggaggtgutggutuygygguttggggttygggutyggutyggutuagguayguygguaggygagutggutuygutattggggtgggyggattggyggygygggyguuatggagauuuuutgtuagtuauygggtggututguuaatuaaaguttgaatuaguayguuaggautatttuaguauutuuuygtgutygauagyguuyguyg

SEQ ID NO: 5 - DNA, 34 bp, Artificial Sequence
  Primer
aggttttgtaaaatattgaagaaaagtttaggag

SEQ ID NO: 6 - DNA, 31 bp, Artificial Sequence
  Primer
caacctcctaataatatacttcaaaccacaa

SEQ ID NO: 7 - DNA, 31 bp, Artificial Sequence
  Primer
tattttggaaaattataagtttattaaggag

SEQ ID NO: 8 - DNA, 33 bp, Artificial Sequence
  Primer
caaaaattaactttaaatcttaaaatccctaac

SEQ ID NO: 9 - DNA, 34 bp, Artificial Sequence
  Primer
aatttttgttagtttgggtataggtttttattat

SEQ ID NO: 10 - DNA, 32 bp, Artificial Sequence
  Primer
accctataacaaaacctactactaaaatctac

SEQ ID NO: 11 - DNA, 30 bp, Artificial Sequence
  Primer
gaattatggtggggtataatagtagggatt

SEQ ID NO: 12 - DNA, 33 bp, Artificial Sequence
  Primer
tctaaacaaacrctcaacttacaattcraattc

SEQ ID NO: 13 - DNA, 27 bp, Artificial Sequence
  STAMP-EP1-Pyroseq-Primer-F
gaagtatattattaggaggttgagaag

SEQ ID NO: 14 - DNA, 26 bp, Artificial Sequence
  STAMP-EP1-Pyroseq-Primer-R(5′-Biotin)
  misc_feature (1)..(26): Wherein R can be g or a
tacrctccttaaaacaatatatcttc

SEQ ID NO: 15 - DNA, 21 bp, Artificial Sequence
  STAMP-EP1-Pyroseq-Primer-Seq
ccctgggacctgtggacaagt

SEQ ID NO: 16 - DNA, 48 bp, Artificial Sequence
  Pyrosequencing detecting sequences
  misc_feature (1)..(48): Wherein Y can be T/U or C
atygagtaaggaagaagttttygttgtygygtattatttgggayggyg